SEQUENCE LISTING (3 sequences)

SEQ ID NO: 1 - protein, 6 aa, Artificial Sequence
  Description of Artificial Sequence Synthetic 6xHis tag
HHHHHH

SEQ ID NO: 2 - DNA, 22 bp, Artificial Sequence
  Description of Artificial Sequence Synthetic oligonucleotide
cggcgatggccctgtgctgccc

SEQ ID NO: 3 - DNA, 25 bp, Artificial Sequence
  Description of Artificial Sequence Synthetic oligonucleotide
caggttcagggggaggtgtgggagg

(19) United States
(12) Patent Application Publication
(10) Pub. No.: US 2016/0264616 A1
(43) Pub. Date: Sep. 15, 2016

(54) THERAPEUTIC COMPOUNDS AND METHODS

(71) Applicant: UNIVERSITY OF IOWA RESEARCH FOUNDATION, Iowa City, IA (US)

(72) Inventors: David L. Roman, Iowa City, IA (US); Duncan Ian Mackie, Iowa City, IA (US)

(73) Assignee: UNIVERSITY OF IOWA RESEARCH FOUNDATION, Iowa City, IA (US)

(21) Appl. No.: 15/066,930

(22) Filed: Mar. 10, 2016

Related U.S. Application Data
(60) Provisional application No. 62/131,061, filed on Mar. 10, 2015.

(51) Int. Cl. C07J 73/00; C07D 311/36; C07D 493/18; C07J 69/00

(52) U.S. Cl. CPC C07J 73/005; C07J 69/00; C07D 311/36; C07D 493/18

(57) ABSTRACT

Regulator of G Protein Signaling (RGS) proteins modulate the complex signaling pathways elicited by G protein coupled receptor activation. Recent studies have implicated RGS proteins in the development and progression of multiple cancers. Provided herein are inhibitors of RGS17, compositions thereof and methods of treating diseases using the inhibitors.

### PRIORITY OF INVENTION

This application claims priority from U.S. Provisional Patent Application No. 62/131,061 filed Mar. 10, 2015, which is hereby incorporated by reference in its entirety.

### GOVERNMENT FUNDING

This invention was made with government support under CA160470 awarded by the National Cancer Institute. The government has certain rights in the invention.

### BACKGROUND

G-protein coupled receptors (GPCRs) are a diverse group of seven transmembrane-spanning receptors that represent targets for over 50% of drugs available on the market (56). These receptors signal through the activation of a heterotrimeric G protein complex, consisting of G α, β, and γ subunits. Upon activation of the receptor, bound guanosine-diphosphate (GDP) is exchanged for guanosine-triphosphate (GTP) in the Gα subunit. This causes a dissociation of the Gα subunit from both the receptor and Gβγ subunit complex, and both the Gα subunit and the Gβγ complex proceed to activate their respective signaling pathways. The signal is terminated by the hydrolysis of GTP to GDP in the Gα subunit (57). The intrinsic, relatively slow rate of hydrolysis of the Gα subunit is temporally modulated by another superfamily of proteins, regulators of G-protein signaling (RGS) proteins, that increases the GTPase rate of a variety of Gα subunits, thus acting as GTPase activating proteins (GAPs) (59).

RGS17 is a member of the A/RZ family of RGS proteins that can induce tumor cell proliferation through the cyclic AMP-PKA-CREB signaling pathway (58). RGS17 has shown that it is localized to the central nervous system, exhibiting prominent neuronal expression in healthy individuals. RGS17's expression pattern changes during pathological states, including being upregulated in both lung and prostate cancers. In these oncogenic states, RGS17 acts to suppress the normal Gαi/o mediated inhibition of adenylyl cyclase. This leads to unregulated adenylyl cyclase activity (i.e. overproduction of cAMP) leading to increased activation of the PKA-CREB signaling pathway. The up regulation of CREB is linked to the differential expression of several strong candidate CREB responsive gene products such as oncogenes FoxP2, CyclinD1, and KCIP1 as well as tumor suppressors FoxO4 and Hnt. Microarray studies determined that the increase in RGS17 increased the expression of a member of the forkhead box P (FoxP1-4) family, FoxP2. The FoxP family members have been implicated in several different oncogenic states as FOXP1 is a tumor suppressor in breast cancer (32) and has been suggested to play a role in prostate cancer (33). FOXP4 expression is down-regulated in kidney cancer (34) and inactivated by translocation in several breast cancer cell lines. In a recent study, FOXP2 has also been implicated with cancer as Campbell et al found FOXP2 overexpression as a strong discriminator between normal lymphocytes and multiple myeloma (35). Further investigation of FoxP2 found that its expression is significantly linked to tumor aggressiveness; especially in non-fusion type prostate cancer (36). Next, Cyclin D1 is a key regulator of the G1 phase progression of the cell cycle (37). Recent studies into Cyclin D1 as a potential therapeutic target for the treatment of cancer found its overexpression to be associated with non-small-cell lung cancer (38-40) as well as metastatic prostate cancer to bone (37). Perhaps more important is the link in vitro and in vivo data provide that indicate a role of sustained overexpression of cyclin D1 in androgen-independent sub-cultured prostate cancer cell lines (41). Another gene affected is KCIP1, Kinase C Inhibitor Protein 1, also known as 14-3-3ε. In lung adenocarcinoma, KCIP1/14-3-3ε was identified as a putative oncogene by a comprehensive functional genomic approach (42). Dysregulation of RGS17 expression also effects the expression of two important tumor suppressor genes, FoxO4 and Hnt (5). FoxO4 encodes the forkhead box protein O4. The FoxO family of transcription factors plays critical roles in a number of physiological and pathological processes including cancer (43). A recent investigation into the role that FoxO4 plays in prostate cancer identified metastasis-suppressor activity through counteracting the PI3K/AKT signaling pathway (44). Also of interest is the newly discovered low expression of the FoxO4 gene in non-small cell lung cancer (45), which is the other cancer type where RGS17 over expression has been implicated for the development of tumors. Further investigation into the role of FoxO4 in non-small cell lung cancer found the loss of FoxO4 correlated with an increase in epithelial-mesenchymal transition. Since all of these significant genes are differential regulated by the loss or gain of RGS17 expression through the PKA-CREB signaling pathway it can be hypothesized that repression of these tumor suppressors, or a combination of this with the activation of CREB-responsive genes may lead to, or may be necessary for the proliferation of tumor cells. An RGS17 inhibitor could act to mitigate the effects of RGS17 up-regulation and return the cAMP-PKA-CREB signaling cascade to normal physiological levels by prolonging the activation of the Gαi/o subunit (46, 47). To this end, our lab hypothesizes that the development of RGS17 specific small molecule inhibitors may be therapeutically beneficial for the treatment of these oncogenic states.

James et al. found that RGS17 expression is enhanced in 80% of lung tumors by an average of 8.3-fold and is also increased 7.5-fold in prostate tumors when compared to patient matched normal tissues. Further investigation of RGS17 demonstrated its ability to control the growth properties of tumor cells. This was evaluated through shRNA-mediated knock-down of the RGS17 transcript in Human H1299 non-small cell lung cancer cells and resulted in a decrease in proliferation over 10 days (5, 6). The in vivo significance of RGS17's effects was demonstrated in athymic nude mice with H1299 human lung cancer cell xenografts which exhibited reduced tumor load and growth when mice were injected with cells that were pretreated with a shRNA directed towards RGS17. Subsequently, RGS17 was identified as a candidate gene for lung cancer (60) and as a susceptibility marker for prostate cancer (61). Interestingly, the association of the RGS17 gene with prostate cancer susceptibility was determined to be 4.34×10⁻¹⁸ (p-value), which represented one of the most significant p values reported in a Genome-wide association study (GWAS). Considering the high association of RGS17 with lung and prostate cancer and that in 2014 lung and prostate cancers will account for an estimated 38% of cancer related deaths in males and lung cancer will account for 26% of all cancer related deaths in females (62) there is a need for inhibitors of Gαo:RGS17 protein:protein interaction.

### SUMMARY OF CERTAIN EMBODIMENTS OF THE INVENTION

A method of treating or preventing a disease or disorder (e.g., cancer (such as prostate cancer, lung cancer, ovarian cancer or liver cancer) or Parkinson's disease) mediated by aberrant G protein signaling comprising administering to a patient (e.g., a human patient) in need thereof a therapeutically effective amount of the compound of formula I (a specific compound of formula I is referred to herein as UI-1956), formula II (also referred to herein as UI-5), formula III (also referred to herein as UI-1590) or formula IV (also referred to herein as UI-1907):

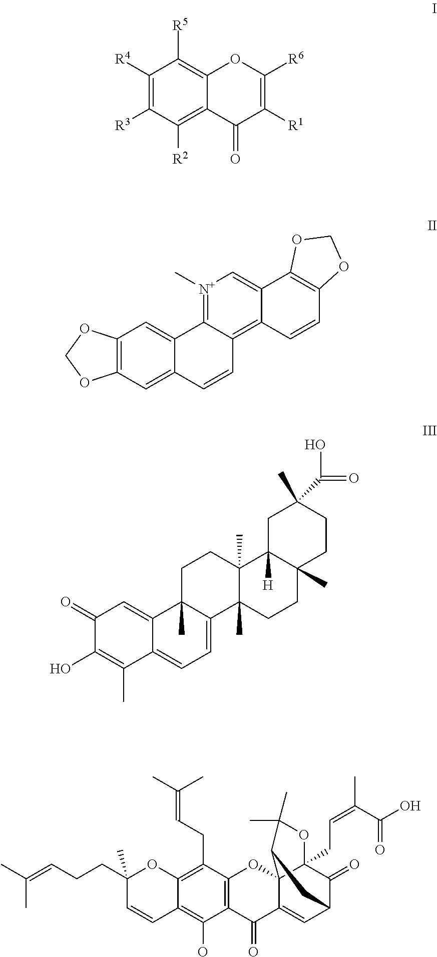

IV

wherein:

R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;

R² is H, halo, (C₁-C₆)alkyl, (C₁-C₆)haloalkyl, —OH, O(C₁-C₆)alkyl or —O(C₁-C₆)haloalkyl;

R³ is H, halo, (C₁-C₆)alkyl, (C₁-C₆)haloalkyl, —OH, —O(C₁-C₆)alkyl or —O(C₁-C₆)haloalkyl;

R⁴ is H, halo, (C₁-C₆)alkyl, (C₁-C₆)haloalkyl, —OH, —O(C₁-C₆)alkyl or —O(C₁-C₆)haloalkyl;

R⁵ is H, halo, (C₁-C₆)alkyl, (C₁-C₆)haloalkyl, —OH, —O(C₁-C₆)alkyl or —O(C₁-C₆)haloalkyl; and

R⁶ is H, halo or (C₁-C₆)alkyl;

or a pharmaceutically acceptable salt thereof.

A method of treating or preventing a disease or disorder (e.g., cancer (such as prostate cancer, lung cancer, ovarian cancer or liver cancer) or Parkinson's disease) comprising administering to a patient (e.g., a human patient) in need thereof a therapeutically effective amount of the compound of formula I (a specific compound of formula I is referred to herein as UI-1956), formula II (also referred to herein as UI-5), formula III (also referred to herein as UI-1590) or formula IV (also referred to herein as UI-1907):

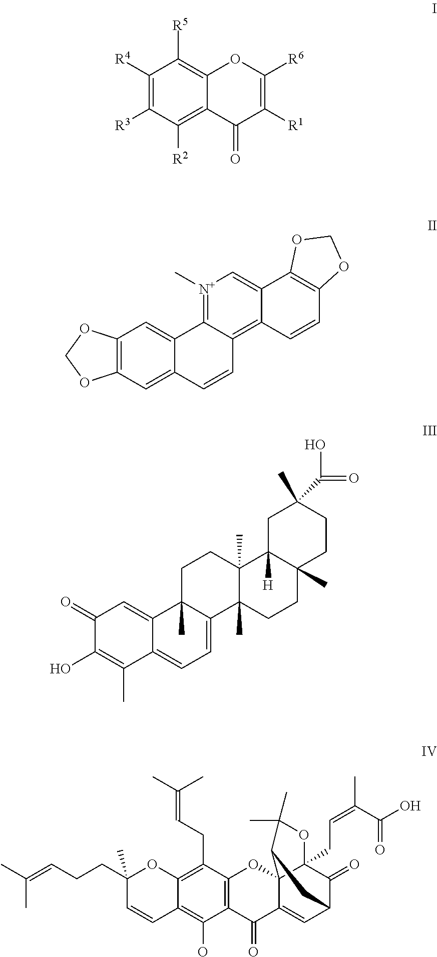

wherein:

R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;

R² is H, halo, (C₁-C₆)alkyl, (C₁-C₆)haloalkyl, —OH, O(C₁-C₆)alkyl or —O(C₁-C₆)haloalkyl;

R³ is H, halo, (C₁-C₆)alkyl, (C₁-C₆)haloalkyl, —OH, —O(C₁-C₆)alkyl or —O(C₁-C₆)haloalkyl;

R⁴ is H, halo, (C₁-C₆)alkyl, (C₁-C₆)haloalkyl, —OH, —O(C₁-C₆)alkyl or —O(C₁-C₆)haloalkyl;

R⁵ is H, halo, (C₁-C₆)alkyl, (C₁-C₆)haloalkyl, —OH, —O(C₁-C₆)alkyl or —O(C₁-C₆)haloalkyl; and

R⁶ is H, halo or (C₁-C₆)alkyl;

or a pharmaceutically acceptable salt thereof.

One embodiment provides a method of treating or preventing a disease or disorder (e.g., cancer (such as prostate cancer, lung cancer, ovarian cancer or liver cancer) or Parkinson's disease) mediated by overexpression of RGS17 comprising administering to a patient (e.g., a human patient) in need thereof and which patient overexpresses RGS17 a therapeutically effective amount of a compound of that inhibits the interaction of RGS17 and Gαo.

One embodiment provides a pharmaceutical composition comprising a compound of formula I, formula II, formula III or formula IV or a pharmaceutically acceptable salt thereof, as described herein, and a pharmaceutically acceptable carrier or excipient.

One embodiment provides novel compounds of formula I:

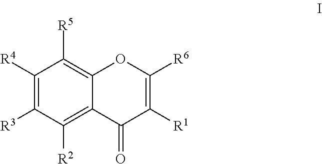

wherein:

R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;

R² is H, halo, (C₁-C₆)alkyl, (C₁-C₆)haloalkyl, —OH, O(C₁-C₆)alkyl or —O(C₁-C₆)haloalkyl;

R³ is H, halo, (C₁-C₆)alkyl, (C₁-C₆)haloalkyl, —OH, —O(C₁-C₆)alkyl or —O(C₁-C₆)haloalkyl;

R⁴ is H, halo, (C₁-C₆)alkyl, (C₁-C₆)haloalkyl, —OH, —O(C₁-C₆)alkyl or —O(C₁-C₆)haloalkyl;

R⁵ is H, halo, (C₁-C₆)alkyl, (C₁-C₆)haloalkyl, —OH, —O(C₁-C₆)alkyl or —O(C₁-C₆)haloalkyl; and

R⁶ is H, halo or (C₁-C₆)alkyl;

or a pharmaceutically acceptable salt thereof;

provided the compound is not 5,6,7-trihydroxy-3-(3,4,5-trihydroxyphenyl)-4H-chromen-4-one or a salt thereof.

One embodiment provides a method of inhibiting the binding interaction of RGS17 to Gαo in a cell in vitro or in vivo comprising, contacting said cell with the compound of formula I, formula II, formula III or formula IV as described herein or salt thereof.

One embodiment provides a method of inhibiting RGS17-accelerated Gαo GTPase activity in a cell in vitro or in vivo, comprising contacting said cell with the compound of formula I, formula II, formula III or formula IV as described herein or salt thereof.

One embodiment provides a method of treating or preventing a disease or disorder (e.g., cancer (such as prostate cancer, lung cancer, ovarian cancer or liver cancer) or Parkinson's disease) mediated by overexpression of RGS17 protein comprising administering to a patient (e.g., a human patient) in need thereof a therapeutically effective amount of the compound UI-5, UI-1590 or UI-1956:

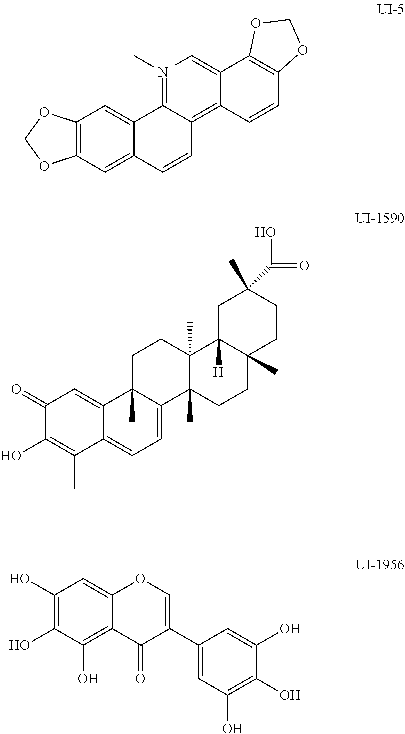

or a pharmaceutically acceptable salt thereof.

One embodiment provides a method of inhibiting the binding interaction of RGS17 to Gαo in a cell comprising contacting said cell with the compound UI-5, UI-1590, UI-1907 or UI-1956:

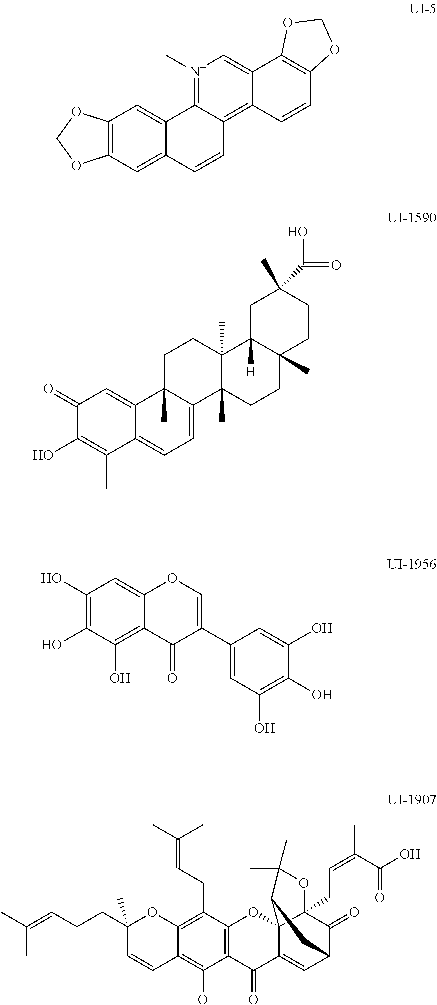

or a pharmaceutically acceptable salt thereof.

One embodiment provides a method of inhibiting RGS17-accelerated Gαo GTPase activity in a cell comprising contacting said cell with the compound UI-5, UI-1590 or UI-1956:

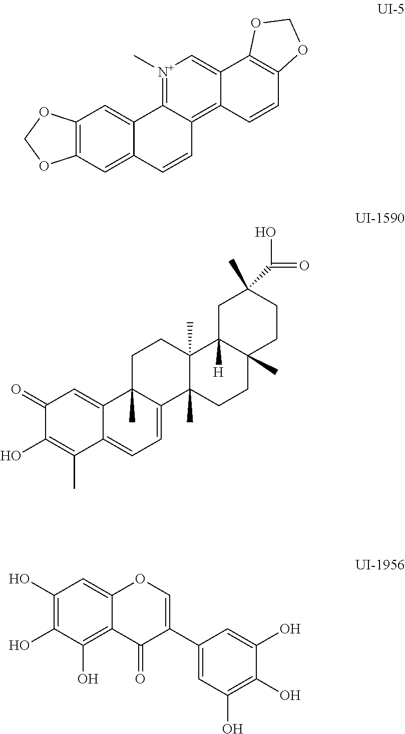

or a pharmaceutically acceptable salt thereof.

### BRIEF DESCRIPTION OF THE FIGURES

FIG. 1 Shows the four chemical structures UI5, UI1590, UI1907 and UI1956.

FIG. 2 Illustrates the determination of the Z factor. Accessing the suitability of the assay for high-throughput screening. In a 1536-well plate, 768 wells were used as a positive control (+AMF) and 768 wells were deprived of guanosine diphosphate (GDP) and AMF, representing the negative control. The signal-to-noise ratio (28), coupled with the Z factor of 0.65, allows for a large screening window for compounds that inhibit the protein:protein interaction (ppi) greater than 50%. CPS, counts per second.

FIG. 3 shows the high-throughput screening of the MicroSource SPECTRUM library. Initial screening of the 2,320 compounds was conducted using two 1536 well plates. This pilot screen yielded 99 initial hits that inhibited the AlphaScreen signal greater than 50%.

FIG. 4A illustrates the determination of the RGS17:Gαo ppi by AlphaScreen. UI-5, UI-1590, UI-1907 and UI-1956 exhibited the ability to inhibit the interaction of RGS17 with Gαo in a dose dependent manner. Data represents n=3, in triplicate.

FIG. 4B illustrates the measurement of protein stability upon ligand binding through Differential Scanning Fluorimetry (DSF). All four compounds were tested for specific binding to the RGS protein versus the Gαo subunit when compared to DMSO controls. UI-5, UI-1590 and UI-1956 were found to stabilize RGS17 against thermal denaturing. UI-1907 was found to bind both the RGS and G protein subunit in a non-specific manner.

FIG. 4C illustrates the inhibition of RGS17's GAP activity with Gαo by malachite Green assay. All four compounds were tested for inhibition of the GTPase acceleration activity of RGS17 in the malachite green steady-state GTPase activity assay. UI-1590 exhibited the most potent activity with an IC50 of 6.359 μM. Data represents n=3, normalized in triplicate.

FIG. 4D illustrates the determination of dissociation constants through Isothermal Titration calorimetry (ITC). UI-5 and UI-1956 were further characterized through ITC and their dissociation constants were calculated to be 1.02 and 0.714 μM, respectively. UI-1590 and UI-1907 were not amendable to ITC due to solubility issues at the increase ligand concentration. UI-5 exhibited a stoichiometry (n) of 0.819 and UI-1956 was determined to have an stoichiometry (n) of 0.333.

FIG. 5A-5F; FIG. 5A illustrates that pAcGFPRGS17FL transfected into HEK293T in the absence of G-protein results in a diffuse pattern of subcellular localization. FIG. 5B show that when pAcGFPRGS17FL is co-transfected with hGαo, RGS17 localizes at the cell membrane. FIGS. 5C and 5D show that when treated with compound UI-5, RGS17FL does not diffuse back in to the cytoplasm over a time course of 0-10 min (C and D respectively). FIG. 5E shows the truncation of RGS17 did not alter the subcellular localization of the fusion protein when transfected in to HEK293T cells. In the absence of G protein, GFPRGS17ΔN is diffuse throughout the cytoplasm. FIG. 5F shows that when truncated GFPRGS17ΔN is co-transfected with hGαo, the fusion protein still localizes at the cell membrane. Scale bars represent 10 μm.

FIG. 6 illustrates HEK293T cells co-transfected with GFPRGS17RH and hGαo were treated with 100 μM UI-5, UI-1590 or UI-1956. All three treatments resulted in delocalization of the fluorescently tagged protein from the membrane, with UI-5 and UI-1590 exhibiting full delocalization after 5 min, and UI-1956 exhibiting full delocalization after 20 min. With successful delocalization observed at 100 μM, lower concentrations were interrogated. All three compounds yielded a delocalization response at a concentration of 10 μM. UI-1590 exhibited full delocalization in 20 min, and UI-5 and UI-1956 exhibited full delocalization after 30 min. Treatment with 1 μM compound did not result in delocalization after 30 min treatment. To ensure that the observed delocalization was compound specific and not a global response to any cell permeable xenobiotic, cells were interrogated with 100 μM CCG-50014. No delocalization was observed out to 30 min. Cells were also treated with a range of DMSO concentrations to see if the compound solvent affected RGS localization, the highest [DMSO] tested is shown here. Over 10 min, DMSO had no effect on RGS localization and largely no effect on overall cell morphology. All experiments were performed in triplicate, with representative images shown above. Scale bars represent 10 μm.

FIG. 7 illustrates exploring the mechanism of action of representative inhibitors. RGS17 was incubated with a 100 μM concentration of inhibitors (UI-5, UI-1590, UI-1907 and UI-1956). Upon completion of the incubation, samples were split into two separate conditions (washed and unwashed) and assayed using AlphaScreen technology. Inhibition by all four compounds was found to be reversible by the restoration of the AlphaScreen signal when compared to vehicle controls (DMSO). Unwashed samples retained the signal suppression by the UI series of small molecule protein:protein interaction inhibitors.

FIG. 8 Matrix-assisted laser desorption ionization-time of flight (MALDI-TOF) evaluation of RGS17 adducts. Compounds were incubated with RGS17 and subjected to interrogation through MALDI-TOF. From this experiment, only the singly and doubly charged species was observed. This indicates the newly discovered inhibitors do not covalently modify RGS17.

### DETAILED DESCRIPTION

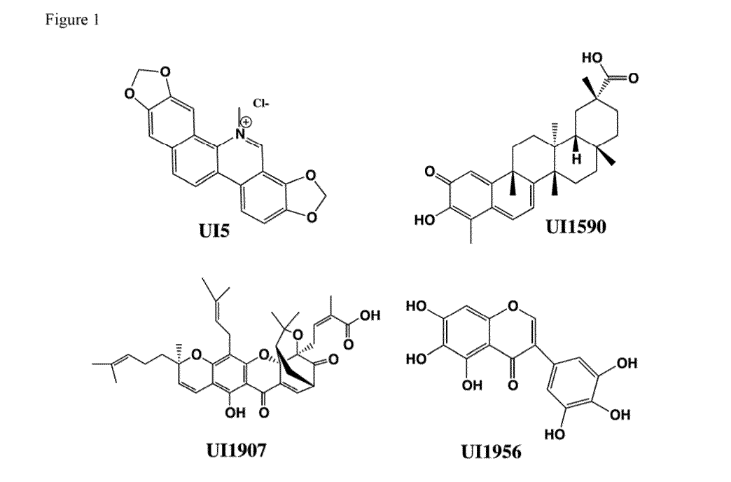

RGS17 is of great interest because its overexpression plays a role in proliferation and metastatic potential of cancers (e.g., prostate cancer, lung cancers). A high-throughput screening campaign focused on identification of small molecules that disrupt the Gαₒ: RGS17 protein:protein interaction such as the three compounds, UI-5, UI-1590 and UI-1956 (FIG. 1) that inhibited the Gαₒ:RGS17 interaction in both biochemical and cell based assays, and exhibit low μM to nM dissociation constants as determined by isothermal titration calorimetry. Most importantly, these inhibitors demonstrated cellular activity, disrupting the Gαₒ:RGS17 interaction in a cellular model.

Accordingly, in an aspect of the invention there is provided a method of treating or preventing a disease or disorder mediated by overexpression of RGS17 protein comprising administering to a patient in need thereof a therapeutically effective amount of the compound UI-5, UI-1590 or UI-1956:

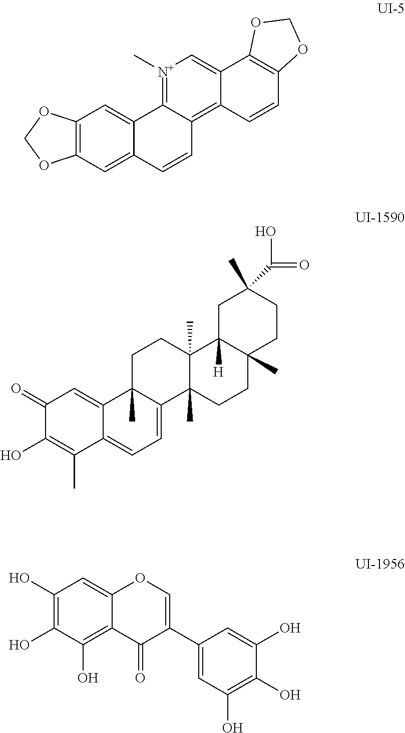

or a pharmaceutically acceptable salt thereof. In one embodiment, disease or disorder is cancer. In one embodiment, the cancer is lung cancer (e.g., non-small cell lung cancer), prostate cancer, ovarian cancer or liver cancer. In one embodiment the disease is Parkinson's disease.

The following definitions are used, unless otherwise described.

The term “alkyl” is a straight or branched saturated hydrocarbon. For example, an alkyl group can have 1 to 8 carbon atoms (i.e., (C₁-C₈)alkyl) or 1 to 6 carbon atoms (i.e., (C₁-C₆ alkyl) or 1 to 4 carbon atoms.

The term “halo” or “halogen” as used herein refers to fluoro, chloro, bromo and iodo.

The term “haloalkyl” as used herein refers to an alkyl as defined herein, wherein one or more hydrogen atoms are each replaced by a halo substituent. For example, a (C₁-C₆)haloalkyl is a (C₁-C₆)alkyl wherein one or more of the hydrogen atoms have been independently replaced by a halo substituent. Such a range includes one halo substituent on the alkyl group to complete halogenation of the alkyl group. The halo substituents may be the same or different

The term “treatment” or “treating,” to the extent it relates to a disease or condition includes inhibiting the disease or condition, eliminating the disease or condition, and/or relieving one or more symptoms of the disease or condition.

The term “patient” as used herein refers to any animal including mammals such as humans, higher non-human primates, rodents domestic and farm animals such as cow, horses, dogs and cats. In one embodiment, the patient is a human patient.

The phrase “therapeutically effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, a mixture containing a stereochemically defined compound is at least 51% of the compound with the absolute stereoisomer depicted. In one embodiment, a mixture containing a stereochemically defined compound is at least 80% of the compound with the absolute stereoisomer depicted. In one embodiment, a mixture containing a stereochemically defined compound is at least 90% of the compound with the absolute stereoisomer depicted. In one embodiment, a mixture containing a stereochemically defined compound is at least 98% of the compound with the absolute stereoisomer depicted.

Specific values listed below for radicals, substituents, and ranges, are for illustration only; they do not exclude other defined values or other values within defined ranges for the radicals and substituents. It is to be understood that one or more values may be combined.

A specific group of compounds are compounds wherein R⁵ and R⁶ are each H.

A specific value for R² is —OH or —O(C₁-C₆)alkyl.

A specific value for R² is —OH.

A specific value for R³ is —OH or —O(C₁-C₆)alkyl.

A specific value for R³ is —OH.

A specific value for R⁴ is —OH or —O(C₁-C₆)alkyl.

A specific value for R⁴ is —OH.

A specific value for R¹ is phenyl substituted with one or more groups independently selected from halo, (C₁-C₆)alkyl, (C₁-C₆)haloalkyl, —OH, —O(C₁-C₆)alkyl or —O(C₁-C₆)haloalkyl.

A specific value for R¹ is phenyl substituted with one or more —OH.

A specific value for R¹ is phenyl substituted with two or more —OH.

A specific value for R¹ is

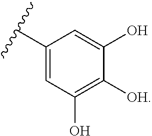 [SMILES: *c1cc(O)c(O)c(O)c1]

A specific compound of formula I is the compound:

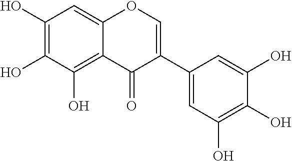 [SMILES: O=c1c(-c2cc(O)c(O)c(O)c2)coc2cc(O)c(O)c(O)c12]

or a pharmaceutically acceptable salt thereof.

Characterization of UI-5, UI-1590 and UI-1956 to determine mechanism of action focused initially on the determination of the potential irreversible modification of the RGS protein by the compounds. Previous work on RGS protein inhibitors widely identified covalent modifying compounds that are profoundly useful for research, are less desirable by their mechanism of action for drug development (13-18). Analysis by matrix-assisted laser desorption ionization-time of flight (MALDI-TOF) mass spectrometry (MS) determined that UI-5, UI-1590 and UI-1956 did not form adducts when incubated with RGS17. These compounds were tested for selectivity versus RGS4 which was chosen as a model protein due to its high level of sensitivity to thiol modification (18). UI-1956 was found to be selective for RGS17 while UI-5 and UI-1590 inhibited both RGS-4 and RGS-17, although both compounds were more selective for RGS17. These inhibitor compounds were also found to disrupt localization of RGS17 to the cellular membrane through association with G protein alpha subunits.

### Miniaturization and Assay Validation

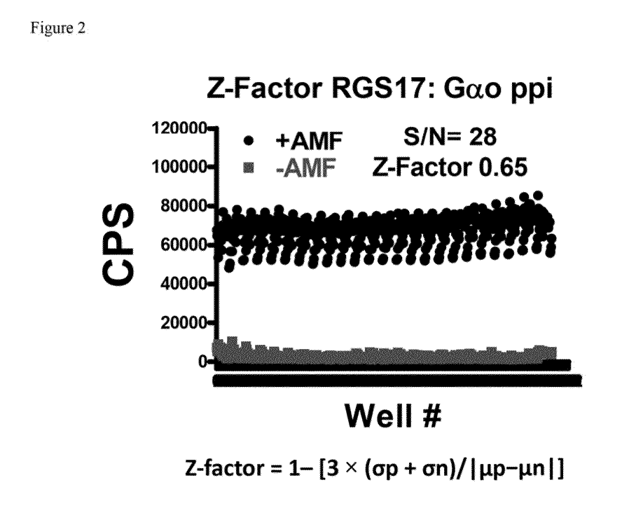

768 wells of a 1536-well plate were used for positive controls in the presence of AMF, which affects the high-affinity Gαo: RGS17 complex, and 768 wells were used as negative controls without AMF in which the formation of Gαo: RGS17 complex is not observed (FIG. 2). The calculated Z factor was determined to be 0.65 with a signal-to-noise ratio of 28. This Z factor is well above the commonly held threshold value of 0.5, indicating a screening paradigm suitable for high-throughput screening (HTS) (19).

### AlphaScreen HTS

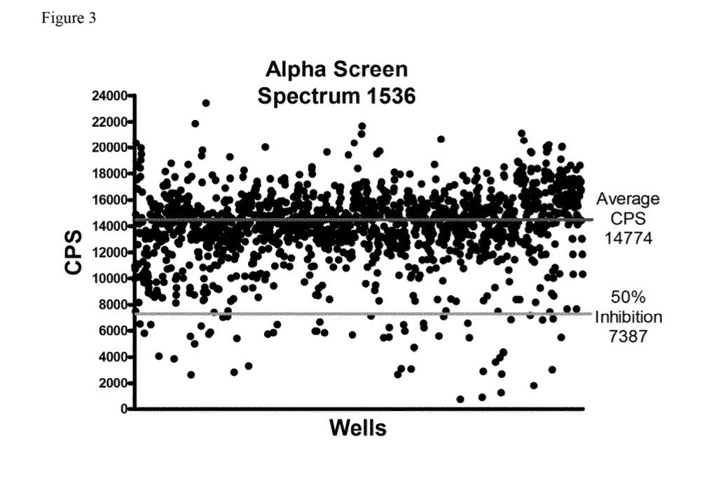

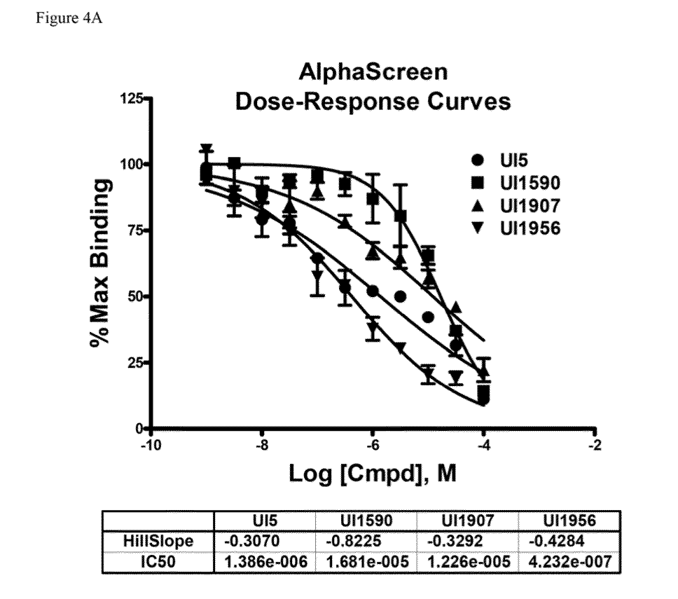

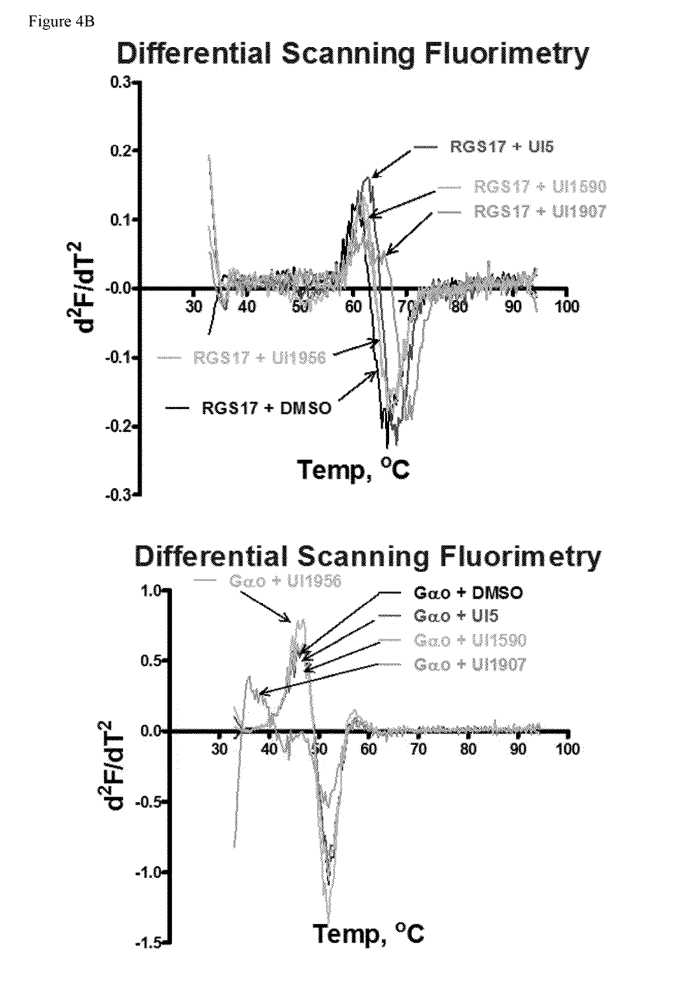

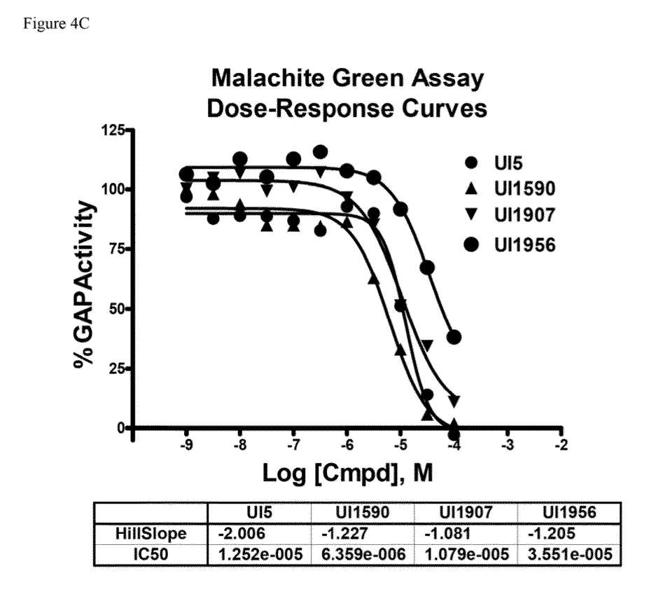

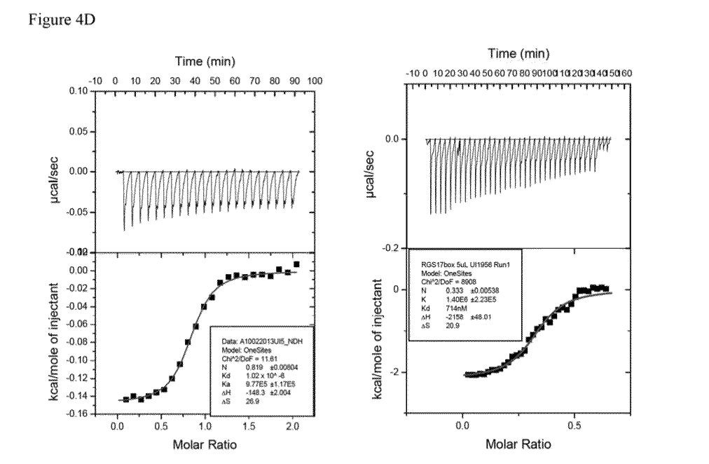

The primary biochemical screen was designed to identify compounds that function as inhibitors of the Gαo:RGS17 ppi. This was accomplished by miniaturizing our previously published HTS paradigm in a 1536-well format (17). This allowed an increase in throughput from 1,000 compounds/hour to over 7,500 compounds/hour. We interrogated the 2320 compound containing MicroSource SPECTRUM Diversity library (Discovery Systems, Inc. Gaylordsville, Conn.) (FIG. 3). We used a cutoff of 50% inhibition or greater to consider a compound as an initial hit. We observed a 4.26% initial hit frequency (99 compounds) with 41 of those compounds confirmed with a single point biotinylated GST counter screen control assay (PE Truehits paradigm) (Table 1). Of these 41 initial hits, seven compounds exhibited an IC50<20 μM (FIG. 4A), yielding a confirmed hit rate of 0.3%. These seven compounds were then analyzed by dose-response control experiments using the non-target control assay which is designated “TrueHits”. Four compounds were found to have no activity in the TrueHits assay when compared to the Gαo:RGS17 ppi (data not shown). These four compounds (FIG. 1) were then chosen for follow-up studies. Accordingly, in an aspect of the invention there is provided a method of inhibiting the binding interaction of RGS17 to Gαo in a cell comprising contacting said cell with the compound UI-5, UI-1590, UI-1907 or UI-1956:

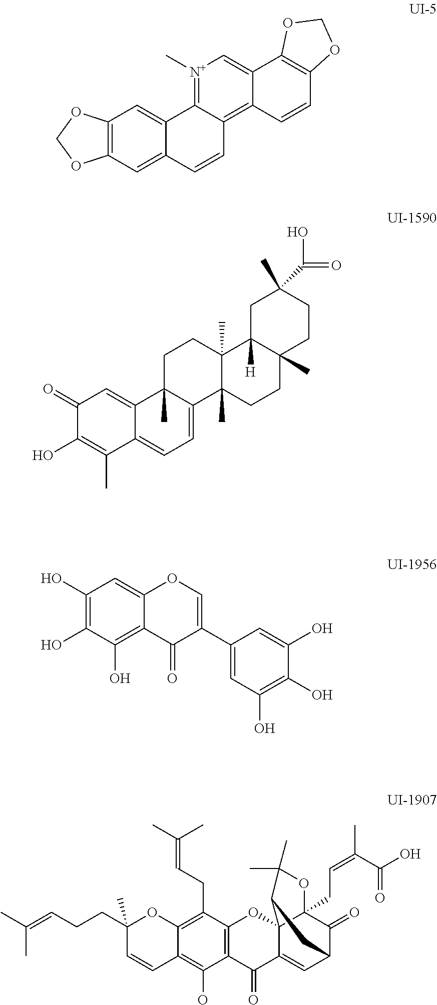

or a pharmaceutically acceptable salt thereof.

### Analysis of Ligand Binding Using Differential Scanning Fluorimetry (DSF)

Further evaluation of the compounds designated UI-5, UI-1590, UI-1907 and UI-1956 was conducted using Differential Scanning Fluorimetry (DSF). This method allows for the rapid measurement of protein stability based on the melting temperature (Tm) of the target protein that is bound by ligand. A shift of Tm indicates a change in protein stability to melting due to stabilization added by the small molecule binding to the target protein. The four compounds were incubated at 50 μM with RGS17 or Gαo in the presence of Sypro Orange. In aqueous solution the fluorescence emission from the dye is very weak. However, when the dye binds to hydrophobic regions of a protein a significant increase in florescence intensity is observed intensity (20). The hydrophobic parts of a native, fully folded, protein in solution are generally buried within the protein and therefore not accessible to the dye resulting in little fluorescence emission. When the protein unfolds the dye molecules can bind to the exposed hydrophobic regions of the proteins, resulting in increased fluorescence. Three of the compounds were found to affect the Tm of RGS17, where UI-1907 was found to alter the thermal stability of both RGS17 and Gαo. UI-1907 can be considered a non-specific RGS17:Gαo protein:protein interaction inhibitor as it affects both binding partners as determined by the activity of UI-1907 to decrease the thermal stability of the Gαo subunit as well as affecting the stability of RGS17. UI-5, UI-1590 and UI-1956 shifted RGS17's Tm by 2.1° C., 1.4° C. and 1.0° C., respectively (FIG. 4B). These three compounds were further characterized for activity against RGS17.

### Inhibition of RGS17 Accelerated Gαo GTPase Activity

Next, the ability of the compounds to inhibit the “GAP” activity of RGS17 was examined. The compounds were tested using a previously described malachite green assay (18), which allows for the detection of free phosphate liberated during the enzymatic cleavage of GTP to GDP by G alpha subunits. In this assay UI-5, UI-1590 and UI-1956 were determined to inhibit RGS17's activity with IC50s of 12 μM, 6 μM and 35 μM, respectively (FIG. 4C). Accordingly, in an aspect of the invention there is provided a method of inhibiting RGS17-accelerated Gαo GTPase activity in a cell comprising contacting said cell with the compound UI-5, UI-1590 or UI-1956:

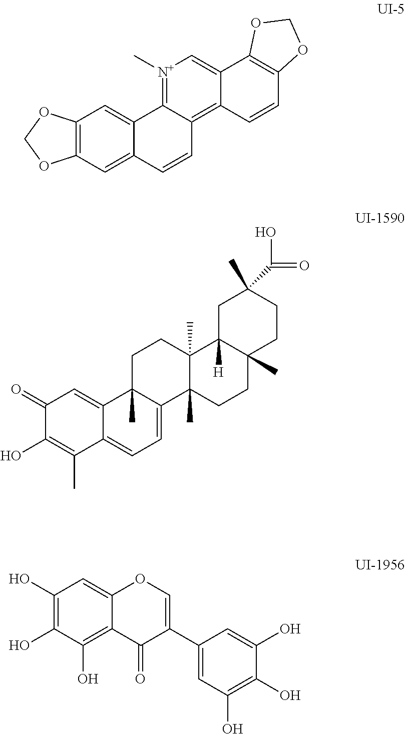

or a pharmaceutically acceptable salt thereof.

### Characterization of the Ligand Binding Properties Using Isothermal Titration Calorimetry (ITC)

ITC was used to determine the binding properties of the lead compounds in solution by determining the dissociation constant (Kd) and stoichiometry (n) of the interaction between RGS17 and the inhibitor compounds. Two compounds, UI-5 and UI-1956, were amenable to ITC and exhibited high affinity for RGS17 with Kd's of 1.02 μM and 714 nM, respectively (FIG. 4D). UI-5 exhibited a stoichiometry of 0.82 while compound UI-1956 was determined to bind with a stoichiometry of 0.33. The corresponding Gibbs free energies of the bindings were −8168 cal/mol for UI-5 and −8389 cal/mol for UI-1956. Decomposing the binding thermodynamics revealed that these two compounds have favorable binding enthalpies. While UI-5 exhibited the lower enthalpy (ΔH=−148 cal/mol), the larger entropic (TΔS=8020.2 cal/mol) component of this compound fully compensated for this significantly lower enthalpy. UI-1956 was determined to have a significantly higher enthalpy component (ΔH=−2158 cal/mol) as well as a lower entropic component (TΔS=6231.3 cal/mol). Interestingly, ITC experiments showed that UI-1956 had the best binding enthalpy and the highest binding affinity of the two compounds. While these two compounds were soluble in the aqueous buffer to the high concentration need to conduct the ITC experiments, two compounds, UI-1590 and UI-1907 were not soluble at the high concentration required for thorough ITC analysis. The results from the four different biochemical techniques led us to further evaluate the mechanism by which these compounds might be eliciting their effects on RGS17.

### Investigation of Compound Reversibility

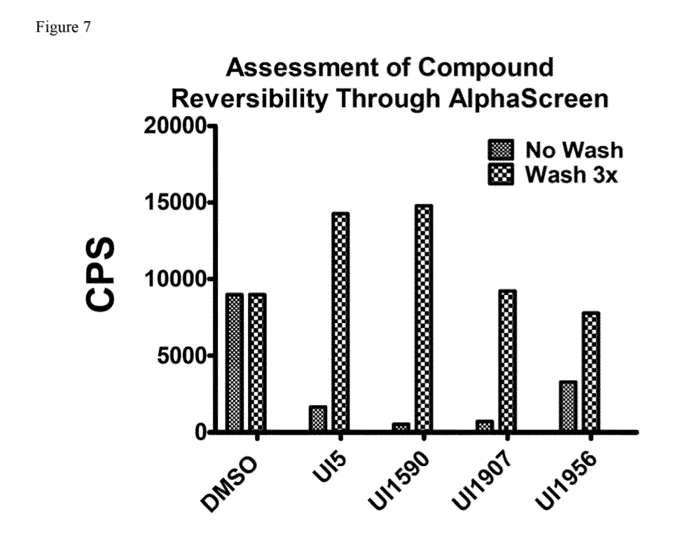

To determine the reversibility of compound inhibition of the RGS17:Gαo protein:protein interaction, RGS17 was treated with 100 μM compound. This concentration was determined through the earlier dose-response experiments to inhibit the AlphaScreen assay >25% for each of the compounds (UI-5, UI-1590, UI-1907 and UI-1956). Upon completion of incubation with compounds, each sample was washed three times with ASB buffer. This method will promote dissociation of any compounds that are non-covalent where as any covalent modifiers would result in persistent protein:protein interaction inhibition. In this experiment, all four compounds were found to fully inhibit the maximum binding of RGS17 to the G alpha subunit (FIG. 7). The corresponding “washed” samples were found to restore binding when compared to vehicle (DMSO) treated and washed samples. This restoration of binding confirms the reversible nature of the UI series of compounds but further validation of non-covalent modification was pursued to confirm these results.

### Evaluation of Protein: Ligand Adducts by Mass Spectrometry

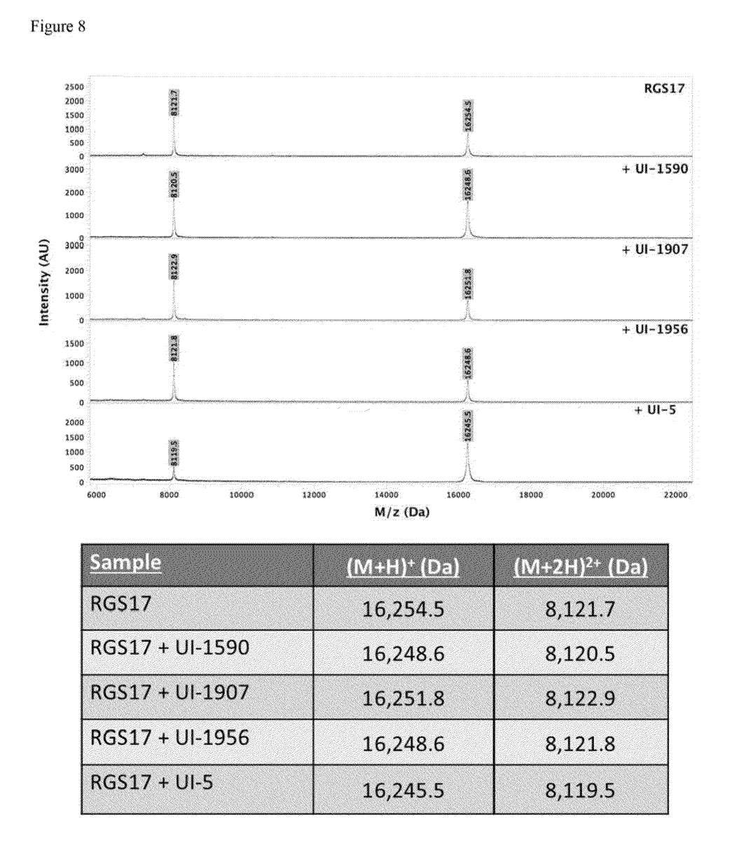

Matrix-assisted laser desorption ionization time of flight (MALDI-TOF) mass spectrometry was employed to evaluate if lead compounds were capable of covalently modifying RGS17's RGS homology domain. Covalent adducts on RGS17 were not detected with any compounds using two-fold molar excess of compound when incubated for one hour with RGS17ΔN (FIG. 8). For all samples, the only species that were observed were singly and doubly charged unmodified, intact protein with mass-to-charge ratios of 8,120 and 16,248 Da, respectively. These data indicate that the compounds identified here inhibit RGS17 GAP activity through a non-covalent mechanism.

RGS17 Translocates to the Cell Membrane when Expressed with Gαo.

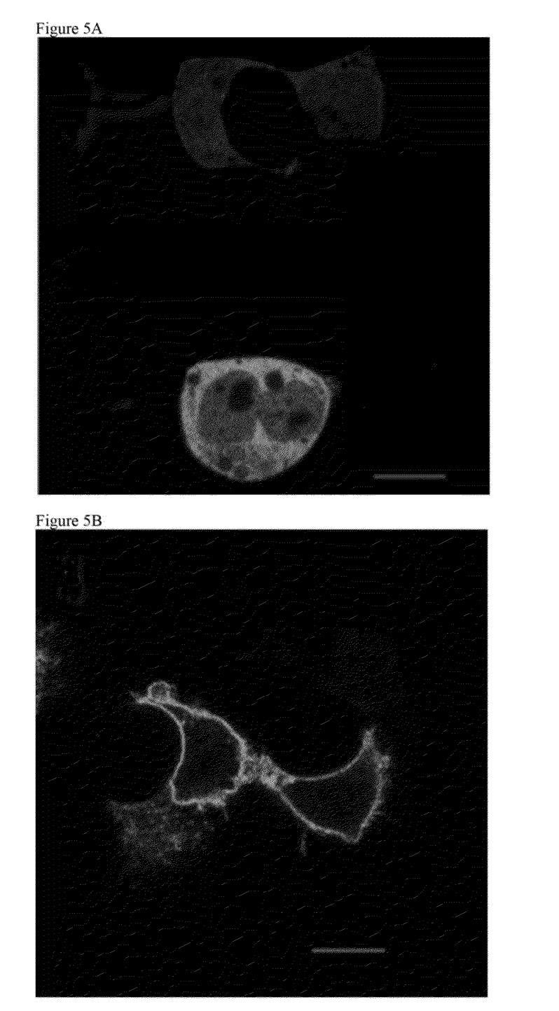

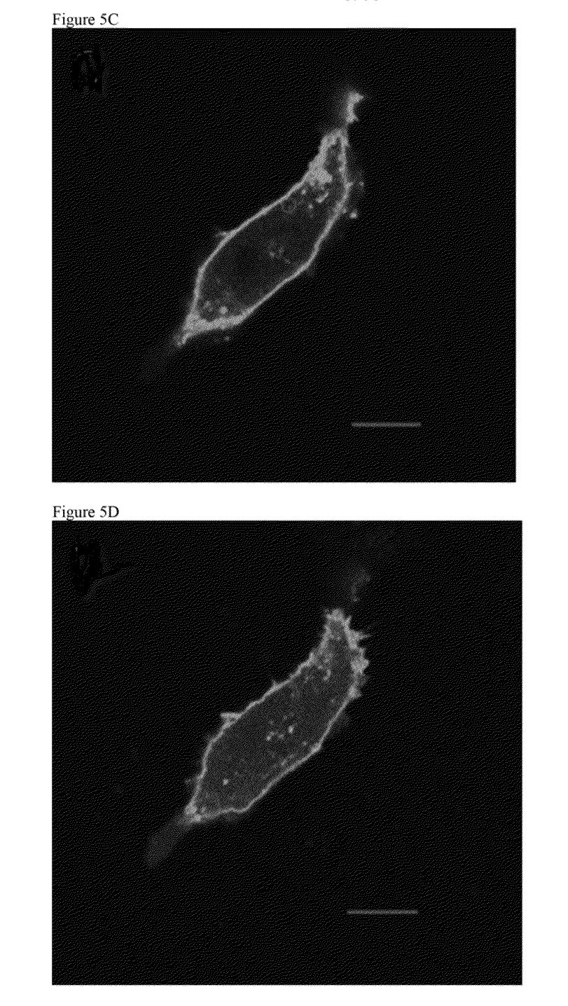

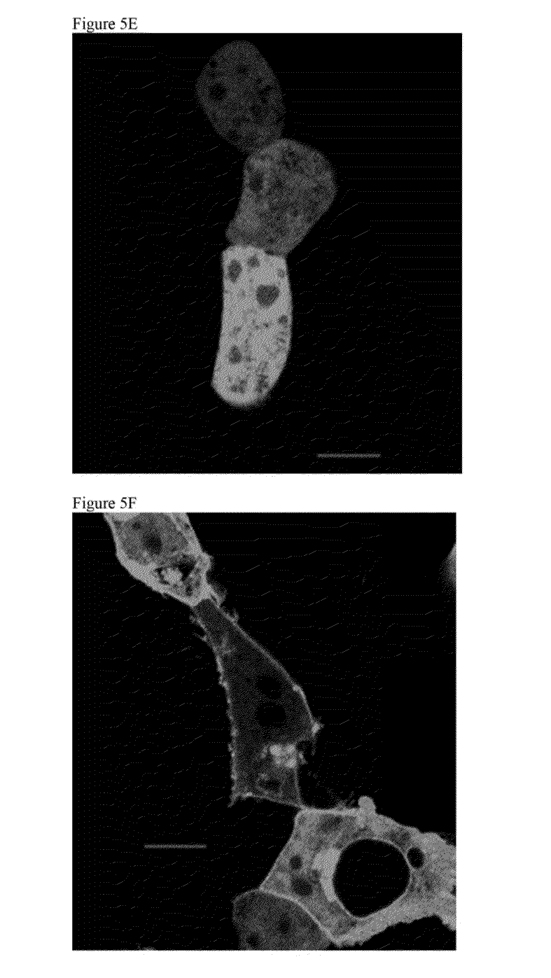

The activity of compounds in a cellular model was next evaluated. Previous studies showed that a different RGS protein, RGS4, localized to the cell membrane when co-transfected with its cognate binding partner Gαo and this interaction could be disrupted with an RGS4 small molecule inhibitor, indicative of activity in cells (21). Previous results show that RGS17 has affinity for Gαo in a biological system, and is thus also amenable for such subcellular localization experiments described above (22). To test this hypothesis, an N-terminal Green Fluorescent Protein (GFP) fusion with full length RGS17 was constructed and cotransfected with human Gαo in HEK293T cells, and subcellular localization of the fusion protein to the plasma membrane was observed via confocal microscopy. (FIG. 5A, 5B). Accordingly, there is provided a method for inhibiting RGS17 translocation to a cell membrane comprising contacting said cell with the compound UI-5, UI-1590 or UI-1956:

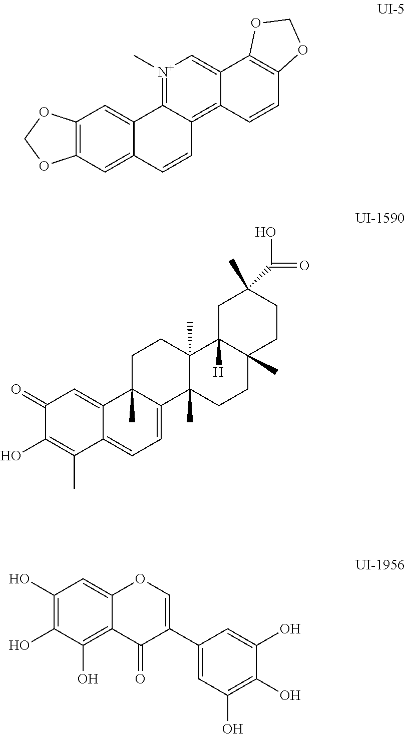

or a pharmaceutically acceptable salt thereof.
Treatment with Inhibitor Compounds Results in RGS17 Delocalization from the Membrane

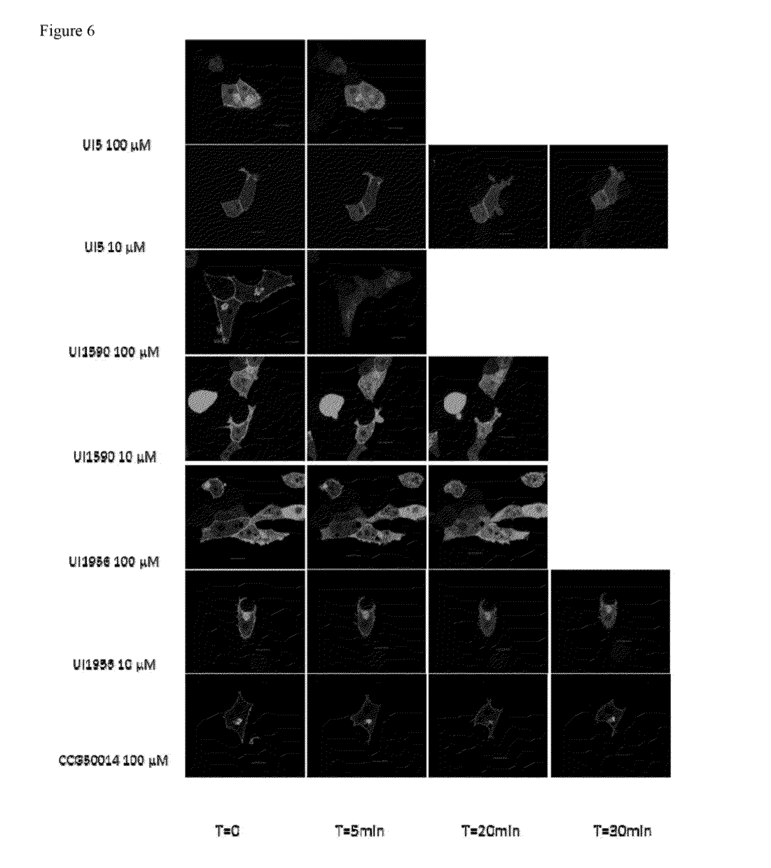

HEK293T cells co-transfected with GFP-RGS17FL and human Gαo were treated with UI-5, UI-1590 and UI-156 to determine if the compounds could disrupt the RGS17:Gαo interaction that could drive RGS17 to the membrane. Gαo is myristoylated and prior studies with RGS4 indicated that the G protein interaction with RGS4 drove the localization of RGS4 to the membrane. Surprisingly, after treatment with 100 μM UI-5 for 10 min, RGS17 remained at the membrane. (FIG. 5C, 5D) Interestingly, the N-terminus of RGS17 is subject to post-translational modification, as it contains a cysteine rich domain which undergoes palmitoylation resulting in the protein being anchored in the membrane once trafficked there (23). We hypothesized that post-translational modification of the N-terminus of RGS17FL resulted in the protein being anchored in the cell membrane via palmitoylation of the cysteine string and as such the RGS17 is unable to delocalize to the cytoplasm upon treatment with compound, even if its interaction with Gαo is disrupted. To test this hypothesis, we constructed a GFP-RGS17 fusion protein with an N-terminal truncation, removing the cysteine-rich motif. This is the same RGS17 construct used in the high throughput screening campaign that is fully active despite the N-terminal truncation. Upon co-transfection with Gαo, RGS17ΔN is recruited to the cell membrane, however, in the absence of overexpressed Gαo, RGS17ΔN is diffusely expressed in cytoplasm (FIG. 5E, 5F). Upon treatment with 100 μM of the compounds, RGS17ΔN delocalizes diffusely to the cytoplasm, indicating that the compounds have activity in cells whereby they disrupt the RGS17:Gα protein interaction (FIG. 6). This delocalization occurs using 100 μM UI-5, UI-1590 and UI-1956 but not UI-1907, which appeared to be generally cytotoxic. Compounds UI-5, UI-1590 and UI-1956 also had cellular activity at 10 μM (FIG. 6). As a negative control, we tested a known RGS4 inhibitor (CCG-50014) in this assay against RGS17ΔN (21). Addition of 50014 did not result in RGS17ΔN delocalization from the membrane (FIG. 6). We also treated cells with DMSO (vehicle, 0.5-2%), which did not affect any changes in RGS localization (FIG. 6). Finally, to ensure the localization was not due to the GFP tag, pAcGFP vector was co-transfected into HEK293T cells with hGαo. GFP was observed as diffuse throughout the cytoplasm, indicating that the localization observed previously was due to the RGS17ΔN:Gαo ppi (data not shown). The delocalization observed at 100 and 10 μM compound was quantified using NIH Image J tool. (Sup. FIG. 3). Cross-sectional fluorescence intensity analysis was used to quantify the intensity of signal at a cross section of the cell. Average signal at the membrane and in the cytoplasm were calculated and change in signal intensity was assessed between time zero and time final. In all cases, there was either a statistically significant decrease in signal from the membrane, or a statistically significant increase in signal in the cytoplasm. In four of six lead compound analyses, both the decrease in membrane signal and the increase in cytosolic signal were statistically significant. Additionally, the control condition of CCG-50014 was subject to the same analyses as the lead compound. In the case of CCG-50014, there was no significant change of signal in either the membrane or cytosolic portions. Accordingly, in an aspect of the invention there is provided a method of delocalizing RGS17 from a cell membrane to the cytoplasm comprising contacting said cell with the compound UI-5, UI-1590 or UI-1956:

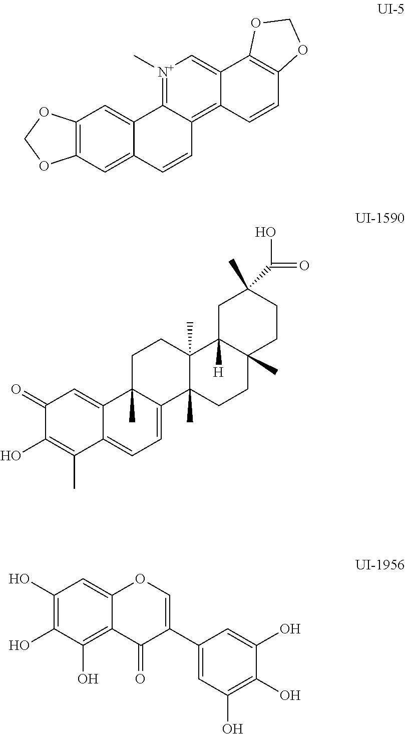

or a pharmaceutically acceptable salt thereof.

The compounds of the invention or used in methods of the invention (the compound of formula I, formula II, formula III or formula IV) may be prepared using established organic synthetic techniques from commercially available starting materials. Alternatively, the compounds may be commercially available.

In cases where compounds are sufficiently basic or acidic, a salt of a compound of formula I, formula II, formula III or formula IV can be useful as an intermediate for isolating or purifying a corresponding compound of formula I, formula II, formula III or formula IV. Additionally, administration of a compound of formula I, formula II, formula III or formula IV as a pharmaceutically acceptable acid or base salt may be appropriate. Examples of pharmaceutically acceptable salts include organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic acid addition salts may also be formed, which include a physiological acceptable anion, for example, chloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Another aspect of the invention provides pharmaceutical compositions or medicaments containing the compounds of the invention or compounds used in the methods of the invention (formula I, formula II, formula III or formula IV) and a therapeutically inert carrier, diluent or excipient, as well as methods of using the compounds of the invention to prepare such compositions and medicaments. In one example, the compounds may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, the compounds are formulated in an acetate buffer, at pH 5. In another embodiment, the compounds are sterile. The compounds may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit RGS protein activity. For example, such amount may be that required to prevent RGS protein from deactivating G protein.

In one example, the pharmaceutically effective amount of the compounds of the invention administered parenterally per dose will be in the range of about 0.001 to 1,000 (e.g., 0.01-100) mg/kg, alternatively about 0.05 to 50 (e.g., 0.1 to 20) mg/kg of patient body weight per day, with the typical initial range of the compounds used being 0.3 to 15 mg/kg/day. In another embodiment, oral unit dosage forms, such as tablets and capsules, preferably contain from about 0.1 to about 1,000 (e.g., 25-100) mg of the compounds of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of formula I, formula II, formula III or formula IV or a pharmaceutically acceptable salt and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1,000 (e.g., 25 mg, 50 mg, 100 mg, 250 mg, or 500 mg) of the compounds of the invention compounded with about 1 to 1,000 (e.g., 90-300) mg anhydrous lactose, about 1 to 100 (e.g., 5-40) mg sodium croscarmellose, about 0.1 to 100 (e.g., 5-30 mg) polyvinylpyrrolidone (PVP) K30, and about 0.1 to 100 (e.g., 1-10 mg) magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compounds, for example 1 to 1000 (e.g., 5-400 mg), of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

One embodiment provides a pharmaceutical composition comprising a compound of formula I, formula II, formula III or formula IV or a stereoisomer or pharmaceutically acceptable salt thereof and a pharmaceutically acceptable carrier. One embodiment provides a pharmaceutical composition comprising a compound of formula I, formula II, formula III or formula IV or a pharmaceutically acceptable salt thereof and a pharmaceutically acceptable carrier for use in the treatment of Parkinson's disease. One embodiment provides a pharmaceutical composition comprising a compound of formula I, formula II, formula III or formula IV or a stereoisomer or pharmaceutically acceptable salt thereof and a pharmaceutically acceptable carrier for use in the treatment of cancer (e.g., prostate cancer, lung cancer, ovarian cancer or liver cancer).

The invention will be more fully understood by reference to the following examples. They should not, however, be construed as limiting the scope of the invention.

### Example 1

### Protein Expression and Purification

Gαo was purified as described previously (17). In brief, 6×-his-tagged Gαo was expressed and purified from transformed B1-21 (DE3) bacteria as described with the exception of 1 mM tris(2 carboxyethyl) phosphine hydrochloride (TCEP) in the buffer in place of 1 mM dithiothreitol (DTT) as the reducing agent. Protein purity was >95% by coomassie staining, and the concentration of active G protein was determined by GTPγ[S]³⁵ binding as described previously (3). RGS17 was purified as previously described (17). This procedure resulted in ˜95% pure RGS17 (20 mg at 1.2 mg/mL).

### Example 2

### Chemical Biotinylation of Gαo

Gαo proteins were biotinylated as previously described using EZ-link Biotin-BMCC (Thermo scientific, Rockford, Ill.) (17). Protein was labeled at a 5:1 biotin: protein ratio following manufacturer protocols. Fractions were pooled and concentrated to 1.66 mg/mL using an ultracel 10 k cutoff centrifugal filter (Millipore, Billerica, Mass.) and protein purity was >95%. The concentration of active G protein was determined by using GTPγS binding as described previously (3).

### Example 3

### Z Factor Calculation in 1536 Well Format

Experiments were performed in Nunc (Thermo scientific, Rockford, Ill.) 1536-well white flat-bottom plates, and samples were read on a Perkin Elmer EnVision Alpha Multimode plate reader. All data were collected and analyzed with Graphpad Prism 6.0 (Graphpad software, San Diego, Calif.).

The 1536-well plates were used to determine the positive and negative control values for the protein interaction assay. In total, 768 wells of the plate were −AMF and represented no protein:protein interaction (background), and 768 wells contained +AMF, which supports the high-affinity protein:protein interaction (ppi) (maximal signal). In total, 144 μL of anti-GST acceptor beads and 240 μL streptavidin donor beads were coupled to RGS17-anti-GST and Gαo-biotin-streptavidin at a 30 nM concentration. This was completed in either 6 mL or 10 mL of assay buffer (AlphaScreen Buffer: ASB) (50 mM HEPES, 100 mM NaCl, 0.1% Lubrol, 1% bovine serum albumin [BSA], pH 8.0). The RGS17-anti-GST coupling was completed in 6 mL while the Gαo-biotin-streptavidin was completed in 10 mL due to the need to split this sample into two set for +AMF and −AMF conditions. The protein/bead mixtures were incubated in the dark on ice for 30 min. Upon completion of coupling, the RGS717-anti-GST bead mixture was resuspended in a total of 12 mL of assay buffer. The Gαo-biotin-streptavidin bead mixture was split into two tubes of 5 mL each. One tube was combined with 10 mL of assay buffer without AMF or GDP for the no-binding control (−AMF). For the positive binding control, the second tube received 10 mL of assay buffer that also contained a final concentration of 50 μM NaF, 50 μM MgCl₂, 50 nM AlCl₃, and 5 μM GDP (+AMF) and was incubated on ice for 10 min. Then, 4.5 μL of the RGS17-anti-GST mixture was added to each well of a total of 1536 wells in a 1536-well plate. In total, 768 wells received 4.5 μL of Gαo-biotin-Streptavidin beads with AMF and 768 wells received 4.5 μL of Gαo-biotin-Streptavidin beads without AMF using a FlexDrop IV (PerkinElmer). The plate was incubated in the dark on 1.5 hr and read at room temperature using the EnVision plate reader.

A Z-factor was calculated using the following equation:

$$Z \text{-} factor = \frac{i - 2 x ( σ p + σ n )}{\vert μ p - μ n \vert} ,$$

where σ represents the standard deviation of positive and negative (binding and nonbinding) (p, n) controls, and μ represents the mean of positive and negative control values. Positive controls were determined using the 768 wells containing AMF and GDP, resulting in a Gαo:RGS17 ppi. The negative controls were determined from the 768 wells that lacked AMF and GDP, resulting in no protein:protein interaction.

### Example 4

### Initial AlphaScreen HTS in Miniaturized Format

In total, 2,320 compounds from the MicroSource SPECTRUM chemical library were screened at a concentration of 40 μM. Two 1536-well plates containing 1,280 compounds and 128 wells of DMSO controls were used. RGS17-anti-GST and Gαo-biotin-streptavidin beads were prepared as previously described. In brief, 100 μg (20 μL) of beads were coupled to 20 ng (10 nM) of each binding partner (Gαo:RGS17) and incubated for 30 min on ice. Then, 4.5 μL of RGS17-anti-GST beads were added using a FlexDrop IV (PerkinElmer, Waltham, Mass.) and incubated for 10 min with compound while the Gαo-biotin-streptavidin beads were incubated with AMF. After incubation, 4.5 μL of the Gαo-biotin-streptavidin bead/protein mixture was added to compound containing wells and incubated on ice for 1.25 hours and read at room temperature using the EnVision plate reader with monochromators.

### Example 5

### Dose-Response Experiments

Experiments were carried out similarly to the high-throughput AlphaScreen assay except this was completed using Corning 384-well white flat bottom plates and the final total volume was 60 μL, with 204 of Gαo-biotin-streptavidin and RGS17-anti-GST beads at a final concentration of 10 nM. Then, 20 μL of compounds in a half log dilution series to yield a final range from 1 nM to 100 μM was added to RGS17-anti-GST beads and incubated for 10 min in the dark on ice. Next, 20 μL of Gαo-biotin-streptavidin beads were then added in the presence of AMF and GDP. Negative controls were determined in the absence of AMF and compound. Maximum binding was determined in the absence of compounds but in the presence of AMF and GDP.

### Example 6

### AlphaScreen Biotinylated GST Counter Screen

Compounds that inhibited the protein:protein interaction with an IC50<20 μM were counter screened in a control assay containing biotinylated GST. This was completed using Corning 384-well white flat bottom plates (Corning, N.Y.). Biotin-GST binds both the anti-GST and streptavidin-coated beads, bringing the beads together artificially and forcing an interaction. Compounds were diluted to yield a range from 1 nM to 100 μM, and 20 μL was added to each well. In 5.28 mL of ASB, 211 ng (42.2 μL) of anti-GST beads was incubated with 300 pM biotin-GST for 30 mins at room temperature. Then, 211 ng (42.2 μL) of streptavidin beads was added and incubated for 30 min on ice. After conjugation was complete, 40 μL of the anti-GST-biotin-GST-streptavidin bead complex was added to each well of compounds (final volume of 60 μL), incubated for 10 min, and read at room temperature on the EnVision plate reader.

### Example 7

### Malachite Green Steady-State GTPase Assay

First, stock solutions of each of the 3 components of the developing solution were prepared according to Monroy et al (18). In brief, Compounds were seeded using a half-log dilution with the highest final concentration of compound at 100 μM down to 1 nM. A final concentration of RGS17 at 1 μM and Gαil at 1 μM was used. A 4×GTP at 1.2 mM diluted in MGB, was used, with a final concentration of 300 μM. To terminate the reaction, 10 μL of a Developing Solution (DS) was 50:12.5:1 (malachite:molybdate:Tween-20) was added to each well using a Microlab Star liquid handling robot (Hamilton Robotics; Reno, Nev.), this achieved a final ratio 4:1 (sample:developing solution) absorbance was read at 642 nm.

### Example 8

### Differential Scanning Fluorimetry

Differential scanning fluorimetry experiments were carried out using white 384-well μltraAMP PCR plates (Sorenson BioSciences; Salt Lake City, Utah). All experiments were carried out as previously described by Phillips and Hernandez de la Pena.(55) In brief, a 1:2000 dilution of Sypro Orange was made by adding 1 μL of Sypro Orange to 2 mL of PBS at pH 7.5. First, 1.2 mg/mL protein was incubated with 50 μM of each compound at room temperature for 15 min in a 10 μL volume. Upon completion of the incubation, 110 μL of the Sypro Orange-PBS solution was added to each compound/protein mixture. This yielded a final volume of 120 μL containing 0.1 mg/mL protein. Finally, 20 μL of the Sypro Orange/compound/protein mix for each compound and RGS17 or Gαo proteins was added to four wells of the 384-well plate. The experiment was run on the Roche LightCycler 480 (Roche, Switzerland) using a two-step method. Starting with a 25° C. baseline step and a second step with a target temperature of 95° C. with continuous acquisition and set acquisition rate of 3 per ° C. All data was collected using the Roche LightCycler data acquisition ability and analyzed with Graphpad Prism 6.0, using first and second derivatives of the fluorescent melting curves.

### Example 9

### Isothermal Titration Calorimetry

RGS17 was concentrated in ITC sample buffer (50 mM HEPES pH 7.5, 100 mM NaCl and 1 mM beta-mercaptoethanol) at 50 μM. Compounds UI-1956 and UI-5 were diluted into ITC sample buffer to reach a final concentration of 500 μM. DMSO concentration in both compound and RGS17 sample was 1%, to account for any DMSO effects. Total injections for UI-1956 were set to 5 μL with a duration time of 10 secs and spacing of 240 secs and for UI-5 were set to 12 μL with a duration time of 24 secs and spacing of 240 secs. The total amount of injections for UI-1956 and UI-5 were, 32 and 23 respectively. All experiments were conducted on a GE MicroCal VP-ITC System (General Electric; Piscataway, N.J.) at 25 C. Heats of dilution were determined by averaging the heat evolved by the last five injections and subtracted from the raw data. The values for affinity, stoichiometry and change in enthalpy were then determined using the ORIGIN software provided by the manufacturer.

### Example 10

### Investigation of Compound Reversibility

First, stock solutions of all four compounds were made at a 3× concentration or 300 μM for a final concentration of 100 μM. The final DMSO concentration in the assay was 1%. Next, RGS17 and Gαₒ were diluted into 300 μL of assay buffer (AlphaScreen Buffer: ASB) (50 mM HEPES, 100 mM NaCl, 0.1% Lubrol, 1% bovine serum albumin [BSA], pH 8.0) at a 3× concentration of 30 nM. Next, 7.2 μL of AlphaScreen beads were added to each separate protein. The protein and bead solution was incubated on ice for 30 mins in the dark. Upon completion of protein:bead conjugation, 600 μL of ASB was added to RGS17. To the Gαₒ sample, 279 μL of AMF (final concentration of 10 μM NaF, 10 μM MgCl₂, 10 nM AlCl₃, and 5 μM GDP) and 321 μL of ASB were added. The RGS17 sample was split into 90 μL tubes and 90 μL of each compound or DMSO vehicle was added to the RGS17:bead mixture and incubated for 15 mins. Upon completion of compound incubation, the RGS17:bead mixture was washed 3 times with 1.8 mL of ASB. The RGS17 protein:bead samples were pelleted after each wash through centrifugation at 15, 000×g for 10 mins. After the final wash step, the RGS17 protein:bead pellet was resuspended in 180 μL of ASB. Next, to a 384 white plate, 30 μL of the RGS17 protein:bead: compound samples either washed or unwashed were added to each well in triplicate. Finally, 15 μL of the Gαo protein:bead sample was added to each well. The plate was incubated at room temperature, in the dark, for 1.25 hours and read on a Synergy2 plate reader (Biotek, Wisnooski, Vt.) with a sensitivity setting of 200, excitation at 680 nm, and emission read at 570 nm. All data was analyzed with Graphpad Prism 6.0. All data was compared to DMSO controls and corrected for possible bead loss due to washing steps.

### Example 11

### Interrogation of Covalent Adduction by MALDI-TOF Mass Spectrometry-MPH

50 μM of indicated compound and 25 μM RGS17RH in 50 mM HEPES pH 7.5, 100 mM NaCl and 1 mM beta-mercaptoethanol were coincubated with in the presence of 1% DMSO for 30 min at ambient temperature followed by a 30 min incubation at 4° C. to allow the formation of covalent adducts. RGS17ΔN in 50 mM HEPES pH 7.5, 100 mM NaCl, 1 mM beta-mercaptoethanol, and 1% DMSO in the absence of compound was used as a negative control. Following treatment with compound, buffer was exchanged for 25 mM ammonium bicarbonate using an Amicon Ultra 10K 0.5 mL centrifugal filtration device (Millipore, Billerica, Mass.). Intact protein molecular weight was then assessed by matrix-assisted laser desorption/ionization-time of flight (MALDI-TOF) mass spectrometry. This was accomplished using a Bruker ultrafleXtreme in the linear TOF mode, employing 384-well Anchor chip targets (Billerica, Mass.). MALDI matrix reagents sinapinic acid and alpha-Cyano-4-hydroxycinnamic acid, purchased from Bruker, were reconstituted to 5 mg/ml in a 1:1 solution of acetonitrile and aqueous 0.1% formic acid (Billerica, Mass.). Samples in ammonium bicarbonate were diluted to 5 μM in aqueous 0.1% formic acid and introduced to target as a 1:1 solution of protein sample to MALDI matrix buffer. After crystal drying, samples were desalted on the target plate using aqueous 0.1% TFA. The laser was configured to emit pulses at 200 Hz at an optimal wavelength of 355 nm, and input energy of 500 μJ/pulse was attenuated 80-90%. Mass scale was calibrated using horse heart myoglobin, and 2000 shots were acquired per spectra.

### Example 12

### Plasmid Construction and Purification

GFP fusion constructs were made using pAcGFP In-Fusion Ready vector (Clontech Catalog No. 632500). Both RGS17 full length (RGS17 FL) and the truncated RGS17 construct (RGS17ΔN) were cloned in using n-terminal Sall and c-terminal EcoRI restriction sites, using the In-Fusion HD Cloning System (Clontech, Catalog No. 638909). Plasmid insertion was confirmed via colony PCR. DNA was transformed in to DH5α E. Coli, and purified via MIDI-Prep (Promega, Ref A2492). Plasmid sequences were confirmed utilizing sequencing services at the University of Iowa (Sanger Sequencing, Iowa Institute of Human Genetics, University of Iowa). Oligo sequences were as follows: Sense strand: 5′-CGGCGATGGCCCTGTGCTGCCC-3′. Antisense strand: 5′-CAGGTTCAGGGGGAGGTGTGGGAGG-3′.

### Example 13

### RGS Localization Assay

Twenty four (24) hours prior to plating, assay plates were coated with 30 μl poly-D-lysine per well. Human Embryonic Kidney 293T cells (HEK293T) were plated in to 96 well tissue culture treated glass bottom view plates (Perkin Elmer, Part No. 6005430) at a density of 25K cells per well. 24 hours post seeding, cells were transfected with hGαₒ in pCDNA 3.1(+), pAcGFPRGS17 FL, pAcGFP RGS17ΔN, empty pAcGFP, hGαₒ+pAcGFPRGS17 FL, hGαₒ+pAcGFP RGS17ΔN, or hGαₒ+empty pAcGFP vector using Lipofectamine 2000 (Life Technologies) as the transfection reagent and according to manufacture protocols. 24 hours post transfection, cell media was removed and replaced with 1000 serum free, phenol red free DMEM (Life Technologies, Ref No. 31053-028). Cells were then imaged using a Zeiss LSM510 confocal microscope (University of Iowa Central Microscopy Core). Images were analyzed using NIH Image J software and Graphpad Prism 6.

### Example 14

### Compound Treatments

Compounds at 2× final desired concentrations were diluted in serum free, phenol red free DMEM. 100 μl compound was added to the assay well, and then cells were observed using the Zeiss LSM510 confocal microscope, with images being acquired at various time points.

### Example 15

### DMSO Control Treatments

2× stocks of DMSO ranging from 0.1-4% DMSO concentration were made in serum free, phenol red free DMEM. 100 μl DMSO solution was added to the assay well for final DMSO concentrations ranging from 0.05-2%. Cells were observed using the Zeiss LSM510 confocal microscope.

### CITATIONS

1. Gilman A G. G proteins: transducers of receptor-generated signals. Annu Rev Biochem. 1987; 56:615-649.
2. Higashijima T, Ferguson K M, Sternweis P C, Smigel M D, Gilman A G. Effects of Mg2+ and the beta gamma-subunit complex on the interactions of guanine nucleotides with G proteins. J Biol Chem. 1987; 262(2):762-766.
3. Sternweis P C, Robishaw J D. Isolation of two proteins with high affinity for guanine nucleotides from membranes of bovine brain. J Biol Chem. 1984; 259(22):13806-13813.
4. Ross E M, Wilkie T M. GTPase-activating proteins for heterotrimeric G proteins: regulators of G protein signaling (RGS) and RGS-like proteins. Annu Rev Biochem. 2000; 69:795-827.
5. James M A, Lu Y, Liu Y, Vikis H G, You M. RGS17, an overexpressed gene in human lung and prostate cancer, induces tumor cell proliferation through the cyclic AMP-PKA-CREB pathway. Cancer Res. 2009; 69(5):2108-2116.
6. You M, Wang D, Liu P, Vikis H, James M, Lu Y, Wang Y, Wang M, Chen Q, Jia D, Liu Y, Wen W, Yang P, Sun Z, Pinney S M, Zheng W, Shu X O, Long J, Gαo Y T, Xiang Y B, Chow W H, Rothman N, Petersen G M, de Andrade M, Wu Y, Cunningham J M, Wiest J S, Fain P R, Schwartz A G, Girard L, Gazdar A, Gaba C, Rothschild H, Mandal D, Coons T, Lee J, Kupert E, Seminara D, Minna J, Bailey-Wilson J E, Amos C I, Anderson M W. Fine mapping of chromosome 6q23-25 region in familial lung cancer families reveals RGS17 as a likely candidate gene. Clin Cancer Res. 2009; 15(8):2666-2674.
7. Hurst J H, Mendpara N, Hooks S B. Regulator of G-protein signalling expression and function in ovarian cancer cell lines. Cell Mol Biol Lett. 2009; 14(1):153-174.
8. Rangel J, Nosrati M, Leong S P, Haqq C, Miller J R, 3rd, Sagebiel R W, Kashani-Sabet M. Novel role for RGS1 in melanoma progression. Am J Surg Pathol. 2008; 32(8):1207-1212.
9. Smalley M J, Iravani M, Leao M, Grigoriadis A, Kendrick H, Dexter T, Fenwick K, Regan J L, Britt K, McDonald S, Lord C J, Mackay A, Ashworth A. Regulator of G-protein signalling 2 mRNA is differentially expressed in mammary epithelial subpopulations and over-expressed in the majority of breast cancers. Breast Cancer Res. 2007; 9(6):R85.
10. Dorsam R T, Gutkind J S. G-protein-coupled receptors and cancer. Nat Rev Cancer. 2007; 7(2):79-94.
11. Eeles R A, Olama A A, Benlloch S, Saunders E J, Leongamornlert D A, Tymrakiewicz M, Ghoussaini M, Luccarini C, Dennis J, Jugurnauth-Little S, Dadaev T, Neal D E, Hamdy F C, Donovan J L, Muir K, Giles G G, Severi G, Wiklund F, Gronberg H, Haiman C A, Schumacher F, Henderson B E, Le Marchand L, Lindstrom S, Kraft P, Hunter D J, Gapstur S, Chanock S J, Berndt S I, Albanes D, Andriole G, Schleutker J, Weischer M, Canzian F, Riboli E, Key T J, Travis R C, Campa D, Ingles S A, John E M, Hayes R B, Pharoah P D, Pashayan N, Khaw K T, Stanford J L, Ostrander E A, Signorello L B, Thibodeau S N, Schaid D, Maier C, Vogel W, Kibel A S, Cybulski C, Lubinski J, Cannon-Albright L, Brenner H, Park J Y, Kaneva R, Batra J, Spurdle A B, Clements J A, Teixeira M R, Dicks E, Lee A, Dunning A M, Baynes C, Conroy D, Maranian M J, Ahmed S, Govindasami K, Guy M, Wilkinson R A, Sawyer E J, Morgan A, Dearnaley D P, Horwich A, Huddart R A, Khoo V S, Parker C C, Van As N J, Woodhouse C J, Thompson A, Dudderidge T, Ogden C, Cooper C S, Lophatananon A, Cox A, Southey M C, Hopper J L, English D R, Aly M, Adolfsson J, Xu J, Zheng S L, Yeager M, Kaaks R, Diver W R, Gaudet M M, Stern M C, Corral R, Joshi A D, Shahabi A, Wahlfors T, Tammela T L, Auvinen A, Virtamo J, Klarskov P, Nordestgaard B G, Roder M A, Nielsen S F, Bojesen S E, Siddiq A, Fitzgerald L M, Kolb S, Kwon E M, Karyadi D M, Blot W J, Zheng W, Cai Q, McDonnell S K, Rinckleb A E, Drake B, Colditz G, Wokolorczyk D, Stephenson R A, Teerlink C, Muller H, Rothenbacher D, Sellers T A, Lin H Y, Slavov C, Mitev V, Lose F, Srinivasan S, Maia S, Paulo P, Lange E, Cooney K A, Antoniou A C, Vincent D, Bacot F, Tessier D C, Initiative CO-CRUG-E, Australian Prostate Cancer B, Oncology UKGPCSCBAoUSSo, Collaborators UKPS, Consortium P, Kote-Jarai Z, Easton D F. Identification of 23 new prostate cancer susceptibility loci using the iCOGS custom genotyping array. Nat Genet. 2013; 45(4):385-391, 391e381-382.
12. Siegel R, Ma J, Zou Z, Jemal A. Cancer statistics, 2014. C A Cancer J Clin. 2014; 64(1):9-29.
13. Roman D L, Ota S, Neubig R R. Polyplexed flow cytometry protein interaction assay: a novel high-throughput screening paradigm for RGS protein inhibitors. J Biomol Screen. 2009; 14(6):610-619.
14. Roman D L. Chapter 11 Identification of Ligands Targeting RGS Proteins High-Throughput Screening and Therapeutic Potential. Prog Mol Biol Transl Sci. 2009; 86:335-356.
15. Blazer L L, Roman D L, Chung A, Larsen M J, Greedy B M, Husbands S M, Neubig R R. Reversible, allosteric small-molecule inhibitors of regulator of G protein signaling proteins. Mol Pharmacol. 2010; 78(3):524-533.
16. Sjogren B, Neubig R R. Thinking outside of the “RGS box”: new approaches to therapeutic targeting of regulators of G protein signaling. Mol Pharmacol. 2010; 78(4):550-557.
17. Mackie D I, Roman D L. Development of a novel high-throughput screen and identification of small-molecule inhibitors of the Galpha-RGS17 protein-protein interaction using AlphaScreen. J Biomol Screen. 2011; 16(8):869-877.
18. Monroy C A, Mackie D I, Roman D L. A high throughput screen for RGS proteins using steady state monitoring of free phosphate formation. PLoS One. 2013; 8(4):e62247.
19. Zhang J H, Chung T D, Oldenburg K R. A Simple Statistical Parameter for Use in Evaluation and Validation of High Throughput Screening Assays. J Biomol Screen. 1999; 4(2):67-73.
20. Lavinder J J, Hari S B, Sullivan B J, Magliery T J. High-throughput thermal scanning: a general, rapid dye-binding thermal shift screen for protein engineering. J Am Chem Soc. 2009; 131(11):3794-3795.
21. Blazer L L, Zhang H, Casey E M, Husbands S M, Neubig R R. A nanomolar-potency small molecule inhibitor of regulator of G-protein signaling proteins. Biochemistry. 2011; 50(15):3181-3192.
22. Mao H, Zhao Q, Daigle M, Ghahremani M H, Chidiac P, Albert P R. RGS17/RGSZ2, a novel regulator of Gi/o, Gz, and Gq signaling. J Biol Chem. 2004; 279(25):26314-26322.
23. De Vries L, Elenko E, Hubler L, Jones T L, Farquhar M G. GAIP is membrane-anchored by palmitoylation and interacts with the activated (GTP-bound) form of G alpha i subunits. Proc Natl Acad Sci USA. 1996; 93(26):15203-15208.
24. Hurst J H, Hooks S B. Regulator of G-protein signaling (RGS) proteins in cancer biology. Biochem Pharmacol. 2009; 78(10):1289-1297.
25. Zhong H, Neubig R R. Regulator of G protein signaling proteins: novel multifunctional drug targets. J Pharmacol Exp Ther. 2001; 297(3):837-845.
26. Neubig R R, Siderovski D P. Regulators of G-protein signalling as new central nervous system drug targets. Nat Rev Drug Discov. 2002; 1(3):187-197.
27. Blazer L L, Neubig R R. Small molecule protein-protein interaction inhibitors as CNS therapeutic agents: current progress and future hurdles. Neuropsychopharmacology. 2009; 34(1):126-141.
28. Traynor J R, Terzi D, Caldarone B J, Zachariou V. RGS9-2: probing an intracellular modulator of behavior as a drug target. Trends Pharmacol Sci. 2009; 30(3):105-111.
29. Nunn C, Mao H, Chidiac P, Albert P R. RGS17/RGSZ2 and the RZ/A family of regulators of G-protein signaling. Semin Cell Dev Biol. 2006; 17(3):390-399.
30. Larminie C, Murdock P, Walhin J P, Duckworth M, Blumer K J, Scheideler M A, Gamier M. Selective expression of regulators of G-protein signaling (RGS) in the human central nervous system. Brain Res Mol Brain Res. 2004; 122(1):24-34.
31. Garzon J, Rodriguez-Munoz M, Lopez-Fando A, Sanchez-Blazquez P. The RGSZ2 protein exists in a complex with mu-opioid receptors and regulates the desensitizing capacity of Gz proteins. Neuropsychopharmacology. 2005; 30(9):1632-1648.
32. Fox S B, Brown P, Han C, Ashe S, Leek R D, Harris A L, Banham A H. Expression of the forkhead transcription factor FOXP1 is associated with estrogen receptor alpha and improved survival in primary human breast carcinomas. Clin Cancer Res. 2004; 10(10):3521-3527.
33. Taylor B S, Schultz N, Hieronymus H, Gopalan A, Xiao Y, Carver B S, Arora V K, Kaushik P, Cerami E, Reva B, Antipin Y, Mitsiades N, Landers T, Dolgalev I, Major J E, Wilson M, Socci N D, Lash A E, Heguy A, Eastham J A, Scher H I, Reuter V E, Scardino P T, Sander C, Sawyers C L, Gerald W L. Integrative genomic profiling of human prostate cancer. Cancer cell. 2010; 18(1):11-22.
34. Teufel A, Wong E A, Mukhopadhyay M, Malik N, Westphal H. FoxP4, a novel forkhead transcription factor. Biochim Biophys Acta. 2003; 1627(2-3):147-152.
35. Campbell A J, Lyne L, Brown P J, Launchbury R J, Bignone P, Chi J, Roncador G, Lawrie C H, Gatter K C, Kusec R, Banham A H. Aberrant expression of the neuronal transcription factor FOXP2 in neoplastic plasma cells. British journal of haematology. 2010; 149(2):221-230.
36. Stumm L, Burkhardt L, Steurer S, Simon R, Adam M, Becker A, Sauter G, Minner S, Schlomm T, Sirma H, Michl U. Strong expression of the neuronal transcription factor FOXP2 is linked to an increased risk of early PSA recurrence in ERG fusion-negative cancers. Journal of clinical pathology. 2013; 66(7):563-568.
37. Drobnjak M, Osman I, Scher H I, Fazzari M, Cordon-Cardo C. Overexpression of cyclin D1 is associated with metastatic prostate cancer to bone. Clin Cancer Res. 2000; 6(5):1891-1895.
38. Gautschi 0, Ratschiller D, Gugger M, Betticher D C, Heighway J. Cyclin D1 in non-small cell lung cancer: a key driver of malignant transformation. Lung cancer. 2007; 55(1):1-14.
39. Santarius T, Shipley J, Brewer D, Stratton M R, Cooper C S. A census of amplified and overexpressed human cancer genes. Nat Rev Cancer. 2010; 10(1):59-64.
40. Li R, An S J, Chen Z H, Zhang G C, Zhu J Q, Nie Q, Xie Z, Guo A L, Mok T S, Wu Y L. Expression of cyclin D1 splice variants is differentially associated with outcome in non-small cell lung cancer patients. Human pathology. 2008; 39(12):1792-1801.
41. Agus D B, Cordon-Cardo C, Fox W, Drobnjak M, Koff A, Golde D W, Scher H I. Prostate cancer cell cycle regulators: response to androgen withdrawal and development of androgen independence. Journal of the National Cancer Institute. 1999; 91(21):1869-1876.
42. Li R, Wang H, Bekele B N, Yin Z, Caraway N P, Katz R L, Stass S A, Jiang F. Identification of putative oncogenes in lung adenocarcinoma by a comprehensive functional genomic approach. Oncogene. 2006; 25(18):2628-2635.
43. Fu Z, Tindall D J. FOXOs, cancer and regulation of apoptosis. Oncogene. 2008; 27(16):2312-2319.
44. Su B, Gao L, Baranowski C, Gillard B, Wang J, Ransom R, Ko H K, Gelman I H. A Genome-Wide RNAi Screen Identifies FOXO4 as a Metastasis-Suppressor through Counteracting PI3K/AKT Signal Pathway in Prostate Cancer. PLoS One. 2014; 9(7):e101411.
45. Xu M M, Mao G X, Liu J, Li J C, Huang H, Liu Y F, Liu J H. Low expression of the FoxO4 gene may contribute to the phenomenon of EMT in non-small cell lung cancer. Asian Pacific journal of cancer prevention: APJCP. 2014; 15(9):4013-4018.
46. Rosenberg D, Groussin L, Jullian E, Perlemoine K, Bertagna X, Bertherat J. Role of the PKA-regulated transcription factor CREB in development and tumorigenesis of endocrine tissues. Ann N Y Acad Sci. 2002; 968:65-74.
47. Shaywitz A J, Greenberg M E. CREB: a stimulus-induced transcription factor activated by a diverse array of extracellular signals. Annu Rev Biochem. 1999; 68:821-861.
48. Bolton J L, Trush M A, Penning T M, Dryhurst G, Monks T J. Role of quinones in toxicology. Chemical research in toxicology. 2000; 13(3):135-160.
49. Dai Y, Desano J, Tang W, Meng X, Meng Y, Burstein E, Lawrence T S, Xu L. Natural proteasome inhibitor celastrol suppresses androgen-independent prostate cancer progression by modulating apoptotic proteins and NF-kappaB. PLoS One. 2010; 5(12):e14153.
50. Ryu Y B, Park S J, Kim Y M, Lee J Y, Seo W D, Chang J S, Park K H, Rho M C, Lee W S. SARS-CoV 3CLpro inhibitory effects of quinone-methide triterpenes from Tripterygium regelii. Bioorg Med Chem Lett. 2010; 20(6):1873-1876.
51. Zhu H, Liu X W, Cai T Y, Cao J, Tu C X, Lu W, He Q J, Yang B. Celastrol acts as a potent antimetastatic agent targeting beta1 integrin and inhibiting cell-extracellular matrix adhesion, in part via the p38 mitogen-activated protein kinase pathway. J Pharmacol Exp Ther. 2010; 334(2):489-499.
52. Lee J H, Choi K J, Seo W D, Jang S Y, Kim M, Lee B W, Kim J Y, Kang S, Park K H, Lee Y S, Bae S. Enhancement of radiation sensitivity in lung cancer cells by celastrol is mediated by inhibition of Hsp90. Int J Mol Med. 2011; 27(3):441-446.
53. Yang H S, Kim J Y, Lee J H, Lee B W, Park K H, Shim K H, Lee M K, Seo K I. Celastrol isolated from Tripterygium regelii induces apoptosis through both caspase-dependent and -independent pathways in human breast cancer cells. Food and chemical toxicology: an international journal published for the British Industrial Biological Research Association. 2011; 49(2):527-532.
54. Barlow C A, Kitiphongspattana K, Siddiqui N, Roe M W, Mossman B T, Lounsbury K M. Protein kinase A-mediated CREB phosphorylation is an oxidant-induced survival pathway in alveolar type II cells. Apoptosis: an international journal on programmed cell death. 2008; 13(5):681-692.
55. Phillips K, de la Pena A H. The combined use of the Thermofluor assay and ThermoQ analytical software for the determination of protein stability and buffer optimization as an aid in protein crystallization. Current protocols in molecular biology/edited by Frederick M Ausubel [et al]. 2011; Chapter 10:Unit10 28.
56. Nürnberg B, Gudermann T, Schultz G (1995) Receptors and G proteins as primary components of transmembrane signal transduction. Journal of Molecular Medicine 73: 123-132.
57. Gilman A G (1987) G proteins: transducers of receptor-generated signals. Annual review of biochemistry 56: 615-649.
58. James M A, Lu Y, Liu Y, Vikis H G, You M. RGS17, an overexpressed gene in human lung and prostate cancer, induces tumor cell proliferation through the cyclic AMP-PKA-CREB pathway. Cancer Res. 2009; 69(5):2108-2116.
59. Berman D M, Gilman A G (1998) Mammalian RGS proteins: barbarians at the gate. The Journal of biological chemistry 273: 1269-1272.
60. You M, Wang D, Liu P, Vikis H, James M, Lu Y, Wang Y, Wang M, Chen Q, Jia D, Liu Y, Wen W, Yang P, Sun Z, Pinney S M, Zheng W, Shu X O, Long J, Gao Y T, Xiang Y B, Chow W H, Rothman N, Petersen G M, de Andrade M, Wu Y, Cunningham J M, Wiest J S, Fain P R, Schwartz A G, Girard L, Gazdar A, Gaba C, Rothschild H, Mandal D, Coons T, Lee J, Kupert E, Seminara D, Minna J, Bailey-Wilson J E, Amos C I, Anderson M W. Fine mapping of chromosome 6q23-25 region in familial lung cancer families reveals RGS17 as a likely candidate gene. Clin Cancer Res. 2009; 15(8):2666-2674.
61. Eeles R A, Olama A A, Benlloch S, Saunders E J, Leongamornlert D A, Tymrakiewicz M, Ghoussaini M, Luccarini C, Dennis J, Jugurnauth-Little S, Dadaev T, Neal D E, Hamdy F C, Donovan J L, Muir K, Giles G G, Severi G, Wiklund F, Gronberg H, Haiman C A, Schumacher F, Henderson B E, Le Marchand L, Lindstrom S, Kraft P, Hunter D J, Gapstur S, Chanock S J, Berndt S I, Albanes D, Andriole G, Schleutker J, Weischer M, Canzian F, Riboli E, Key T J, Travis R C, Campa D, Ingles S A, John E M, Hayes R B, Pharoah P D, Pashayan N, Khaw K T, Stanford J L, Ostrander E A, Signorello L B, Thibodeau S N, Schaid D, Maier C, Vogel W, Kibel A S, Cybulski C, Lubinski J, Cannon-Albright L, Brenner H, Park J Y, Kaneva R, Batra J, Spurdle A B, Clements J A, Teixeira M R, Dicks E, Lee A, Dunning A M, Baynes C, Conroy D, Maranian M J, Ahmed S, Govindasami K, Guy M, Wilkinson R A, Sawyer E J, Morgan A, Dearnaley D P, Horwich A, Huddart R A, Khoo V S, Parker C C, Van As N J, Woodhouse C J, Thompson A, Dudderidge T, Ogden C, Cooper C S, Lophatananon A, Cox A, Southey M C, Hopper J L, English D R, Aly M, Adolfsson J, Xu J, Zheng S L, Yeager M, Kaaks R, Diver W R, Gaudet M M, Stem M C, Corral R, Joshi A D, Shahabi A, Wahlfors T, Tammela T L, Auvinen A, Virtamo J, Klarskov P, Nordestgaard B G, Roder M A, Nielsen S F, Bojesen S E, Siddiq A, Fitzgerald L M, Kolb S, Kwon E M, Karyadi D M, Blot W J, Zheng W, Cai Q, McDonnell S K, Rinckleb A E, Drake B, Colditz G, Wokolorczyk D, Stephenson R A, Teerlink C, Muller H, Rothenbacher D, Sellers T A, Lin H Y, Slavov C, Mitev V, Lose F, Srinivasan S, Maia S, Paulo P, Lange E, Cooney K A, Antoniou A C, Vincent D, Bacot F, Tessier D C, Initiative CO-CRUG-E, Australian Prostate Cancer B, Oncology UKGPCSCBAoUSSo, Collaborators UKPS, Consortium P, Kote-Jarai Z, Easton D F. Identification of 23 new prostate cancer susceptibility loci using the iCOGS custom genotyping array. Nat Genet. 2013; 45(4):385-391, 391e381-382.
62. Siegel R, Ma J, Zou Z, Jemal A. Cancer statistics, 2014. CA Cancer J Clin. 2014; 64(1):9-29.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwiseindicated herein or otherwise clearly contradicted by context.

1. A method of treating or preventing a disease or disorder mediated by aberrant G protein signaling comprising administering to a patient in need thereof a therapeutically effective amount of a compound of formula I, formula II, formula III or formula IV: 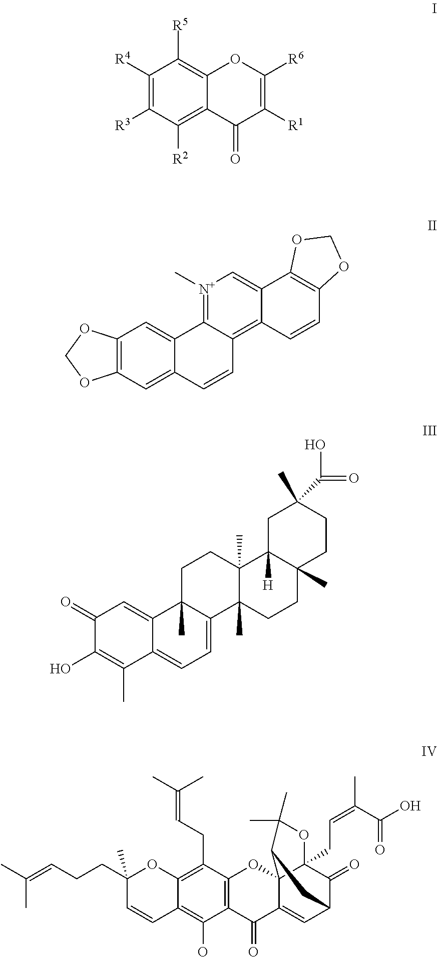
wherein:
R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;
R² is H, halo, (C₁-C₆)alkyl, (C₁-C₆)haloalkyl, —OH, O(C₁-C₆)alkyl or —O(C₁-C₆)haloalkyl;
R³ is H, halo, (C₁-C₆)alkyl, (C₁-C₆)haloalkyl, —OH, —O(C₁-C₆)alkyl or —O(C₁-C₆)haloalkyl;
R⁴ is H, halo, (C₁-C₆)alkyl, (C₁-C₆)haloalkyl, —OH, —O(C₁-C₆)alkyl or —O(C₁-C₆)haloalkyl;
R⁵ is H, halo, (C₁-C₆)alkyl, (C₁-C₆)haloalkyl, —OH, —O(C₁-C₆)alkyl or —O(C₁-C₆)haloalkyl; and
R⁶ is H, halo or (C₁-C₆)alkyl;
or a pharmaceutically acceptable salt thereof.

2. The method of claim 1 wherein the compound is the compound of formula I or a pharmaceutically acceptable salt thereof.

3. The method of claim 1, wherein R⁵ and R⁶ are each H.

4. The method of claim 1, wherein R² is —OH or —O(C₁-C₆)alkyl.

5. (canceled)

6. The method of claim 1, wherein R³ is —OH or —O(C₁-C₆)alkyl.

7. (canceled)

8. The method of claim 1, wherein R⁴ is —OH or —O(C₁-C₆)alkyl.

9. (canceled)

10. The method of claim 1, wherein R¹ is phenyl substituted with one or more groups independently selected from halo, (C₁-C₆)alkyl, (C₁-C₆)haloalkyl, —OH, —O(C₁-C₆)alkyl or —O(C₁-C₆)haloalkyl.

11. The method of claim 1, wherein R¹ is phenyl substituted with one or more —OH.

12-13. (canceled)

14. The method of claim 1 wherein the compound of formula I is the compound: 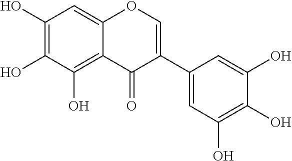 [SMILES: O=c1c(-c2cc(O)c(O)c(O)c2)coc2cc(O)c(O)c(O)c12]
or a pharmaceutically acceptable salt thereof.

15. The method of claim 1, wherein the aberrant G protein signaling is caused by increased RGS activity.

16. The method of claim 1, wherein the aberrant G protein signaling is caused by overexpression of RGS proteins.

17. The method of claim 1, wherein the RGS protein is RGS17.

18. A method of treating or preventing a disease or disorder mediated by overexpression of RGS17 comprising administering to a patient in need thereof and which patient overexpresses RGS17 a therapeutically effective amount of a compound of that inhibits the interaction of RGS17 and Gαo

19. The method of claim 18, wherein the compound is a compound of formula I, formula II, formula III or formula IV: 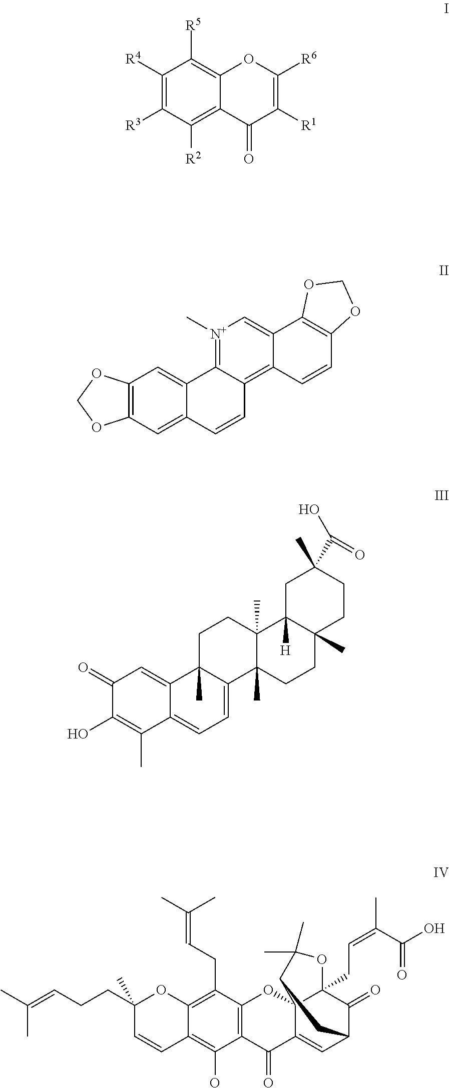
wherein:
R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;
R² is H, halo, (C₁-C₆)alkyl, (C₁-C₆)haloalkyl, —OH, O(C₁-C₆)alkyl or —O(C₁-C₆)haloalkyl;
R³ is H, halo, (C₁-C₆)alkyl, (C₁-C₆)haloalkyl, —OH, —O(C₁-C₆)alkyl or —O(C₁-C₆)haloalkyl;
R⁴ is H, halo, (C₁-C₆)alkyl, (C₁-C₆)haloalkyl, —OH, —O(C₁-C₆)alkyl or —O(C₁-C₆)haloalkyl;
R⁵ is H, halo, (C₁-C₆)alkyl, (C₁-C₆)haloalkyl, —OH, —O(C₁-C₆)alkyl or —O(C₁-C₆)haloalkyl; and
R⁶ is H, halo or (C₁-C₆)alkyl;
or a pharmaceutically acceptable salt thereof.

20. The method of claim 1, wherein the disease or disorder is cancer or Parkinson's disease.

21. (canceled)

22. A pharmaceutical composition comprising a compound of formula I, formula II, formula III or formula IV or a pharmaceutically acceptable salt thereof, as described in claim 1, and a pharmaceutically acceptable carrier or excipient.

23. The method of claim 20, wherein the cancer is prostate cancer, lung cancer, ovarian cancer or liver cancer.

24. A method of inhibiting the binding interaction of RGS17 to Gαo in a cell in vitro or in vivo comprising contacting said cell with the compound of formula I, formula II, formula III or formula IV: 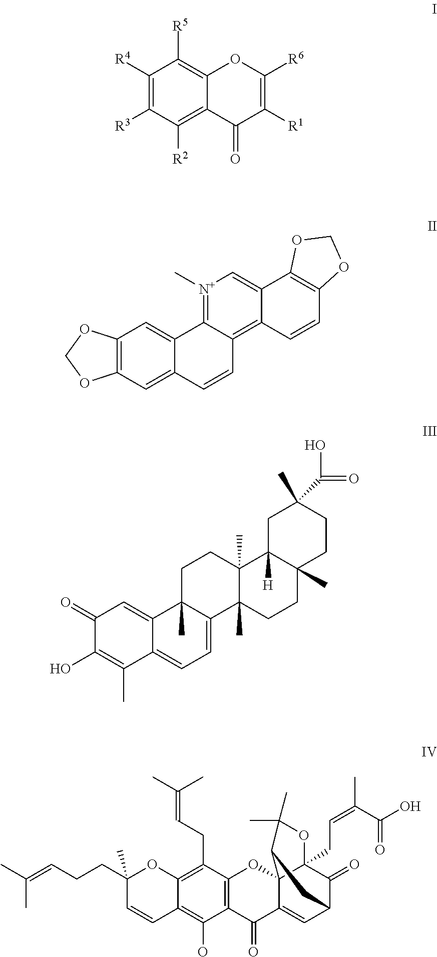
wherein:
R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;
R² is H, halo, (C₁-C₆)alkyl, (C₁-C₆)haloalkyl, —OH, O(C₁-C₆)alkyl or —O(C₁-C₆)haloalkyl;
R³ is H, halo, (C₁-C₆)alkyl, (C₁-C₆)haloalkyl, —OH, —O(C₁-C₆)alkyl or —O(C₁-C₆)haloalkyl;
R⁴ is H, halo, (C₁-C₆)alkyl, (C₁-C₆)haloalkyl, —OH, —O(C₁-C₆)alkyl or —O(C₁-C₆)haloalkyl;
R⁵ is H, halo, (C₁-C₆)alkyl, (C₁-C₆)haloalkyl, —OH, —O(C₁-C₆)alkyl or —O(C₁-C₆)haloalkyl; and
R⁶ is H, halo or (C₁-C₆)alkyl;
or salt thereof.

25. A method of inhibiting RGS17-accelerated Gαo GTPase activity in a cell in vitro or in vivo comprising contacting said cell with the compound of formula I, formula II, formula III or formula IV: 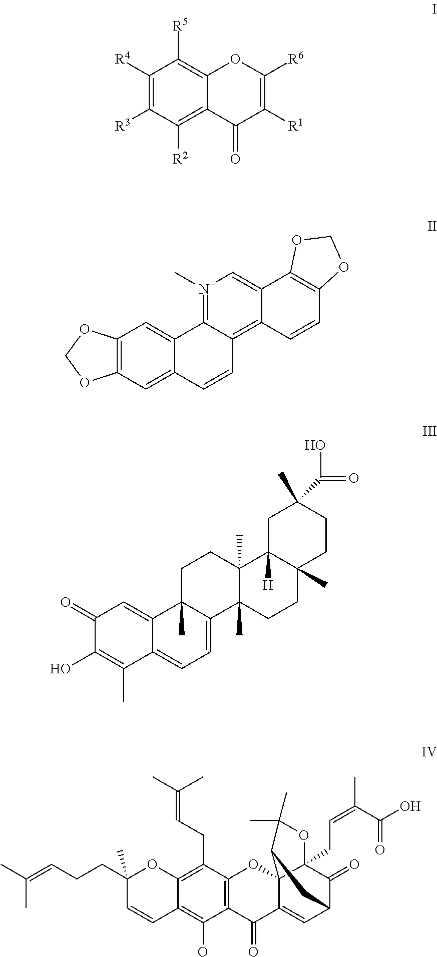
wherein:
R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;
R² is H, halo, (C₁-C₆)alkyl, (C₁-C₆)haloalkyl, —OH, O(C₁-C₆)alkyl or —O(C₁-C₆)haloalkyl;
R³ is H, halo, (C₁-C₆)alkyl, (C₁-C₆)haloalkyl, —OH, —O(C₁-C₆)alkyl or —O(C₁-C₆)haloalkyl;
R⁴ is H, halo, (C₁-C₆)alkyl, (C₁-C₆)haloalkyl, —OH, —O(C₁-C₆)alkyl or —O(C₁-C₆)haloalkyl;
R⁵ is H, halo, (C₁-C₆)alkyl, (C₁-C₆)haloalkyl, —OH, —O(C₁-C₆)alkyl or —O(C₁-C₆)haloalkyl; and
R⁶ is H, halo or (C₁-C₆)alkyl;
or salt thereof.

26. A compound of formula I: 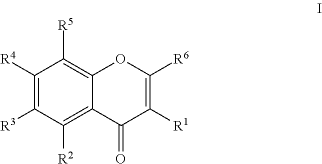
wherein:
R¹ is phenyl optionally substituted with one or more groups independently selected from halo, (C₁-C₆)alkyl, (C₁-C₆)haloalkyl, —OH, —O(C₁-C₆)alkyl and —O(C₁-C₆)haloalkyl;
R² is H, halo, (C₁-C₆)alkyl, (C₁-C₆)haloalkyl, —OH, O(C₁-C₆)alkyl or —O(C₁-C₆)haloalkyl;
R³ is H, halo, (C₁-C₆)alkyl, (C₁-C₆)haloalkyl, —OH, —O(C₁-C₆)alkyl or —O(C₁-C₆)haloalkyl;
R⁴ is H, halo, (C₁-C₆)alkyl, (C₁-C₆)haloalkyl, —OH, —O(C₁-C₆)alkyl or —O(C₁-C₆)haloalkyl;
R⁵ is H, halo, (C₁-C₆)alkyl, (C₁-C₆)haloalkyl, —OH, —O(C₁-C₆)alkyl or —O(C₁-C₆)haloalkyl; and
R⁶ is H, halo or (C₁-C₆)alkyl;
or a pharmaceutically acceptable salt thereof;
provided the compound is not 5,6,7-trihydroxy-3-(3,4,5-trihydroxyphenyl)-4H-chromen-4-one or a salt thereof.

27. The method of claim 18, wherein the disease or disorder is cancer or Parkinson's disease.
